(19) United States
(12) Patent Application Publication
(10) Pub. No.: US 2014/0011222 A1
(43) Pub. Date: Jan. 9, 2014

(54) MULTIPLE MECHANISMS FOR MODULATION OF THE P13 KINASE PATHWAY

(71) Applicant: NODALITY, INC., South San Francisco, CA (US)

(72) Inventor: Wendy J. Fantl, San Francisco, CA (US)

(73) Assignee: NODALITY, INC., South San Francisco, CA (US)

(21) Appl. No.: 13/900,170

(22) Filed: May 22, 2013

Related U.S. Application Data
(63) Continuation of application No. 12/703,741, filed on Feb. 10, 2010, now abandoned.
(60) Provisional application No. 61/157,900, filed on Mar. 5, 2009.
(60) Provisional application No. 61/151,387, filed on Feb. 10, 2009.

(51) Int. Cl. C12Q 1/48

(52) U.S. Cl. CPC C12Q 1/485
USPC 435/15

(57) ABSTRACT

An embodiment of the present invention is a method for measuring the post translational states and expression levels of proteins in the PI3K and/or mTOR for use in diagnosis, prognosis and drug screening applications.

### CROSS-REFERENCE

This application is a continuation of U.S. patent application Ser. No. 12/703,741 filed Feb. 10, 2010, which claims the benefit of priority to U.S. Provisional Application No. 61/157,900, filed Mar. 5, 2009, and U.S. Provisional Application No. 61/151,387, filed on Feb. 10, 2009, which applications are incorporated herein by reference.

### BACKGROUND OF THE INVENTION

Many conditions are characterized by disruptions of cellular pathways that lead, for example, to aberrant control of cellular processes, with uncontrolled growth and increased cell survival. These disruptions are often caused by changes in the activity of molecules participating in cellular pathways. For example, alterations in specific signaling pathways have been described for many cancers.

Elucidation of the signal-transduction networks that drive neoplastic transformation in both solid tumors and hematological cancers has led to rationally designed cancer therapeutics that target signaling molecules. Accordingly, there is a need to look at cell populations to determine what signaling events may contribute to their responses to compounds.

Phosphoinositide 3-kinases (PI 3-kinases or PI3Ks) are a family of enzymes involved in cellular functions such as cell growth, proliferation, differentiation, motility, survival and intracellular trafficking, which in turn are involved in a variety of disorders, including cancer, immune disorders, and diabetes. Thus, there remains a need for improved methods of measuring phosphorylation levels of PI3K, mTOR, and other proteins in the PI3K and/or mTOR pathway.

Mammalian target of rapamycin (mTOR) is a serine/threonine protein kinase that regulates cell growth, cell proliferation, cell motility, cell survival, protein synthesis, and transcription. mTOR exists in two complexes, mTORC1 and mTORC2 (Guertin and Sabatini (2009) Science Signaling 2:1-6). mTOR regulates signals from upstream pathways, including insulin, growth factors (such as IGF-1 and IGF-2), and mitogens. The mTOR pathway is involved in human disorders, such as cancer and age-related diseases, as well as immune-related conditions, such as transplant rejection.

### SUMMARY OF THE INVENTION

In some embodiments, the method provides for a diagnosis of a condition, such as cancer, immune disorder or diabetes. In other embodiments, the method provides for the classification of a condition, such as cancer. Classification can include, but is not limited to, a determination of disease subtype, disease stage, disease activity level, and responsiveness to treatment. In other embodiments, a prediction is made as to the likelihood of a possible disease-related outcome. In one embodiment, the prediction relates to the future course of disease activity. The future course of predicted activity may be a change, as in an increase or decrease in disease activity, or the future course of activity may be predicted to remain substantially similar to the activity level at the time of analysis.

In some embodiments, a prediction is made as to the likelihood of responsiveness to one or more treatments. Treatments about which a prediction can be made can include a treatment the subject has not yet received, or changes in treatments with which a subject is being treated. Changes can include, but are not limited to changes in dose, dosing schedule, route of administration, and/or combination with additional treatments. In some embodiments, a prediction guides the selection of or changes in treatment received by a subject.

In some embodiments, the at least one modulator with which a cell from a subject is contacted is a therapeutic agent, wherein the therapeutic agent is one used to treat a condition, such as cancer.

In some embodiments, the invention is a method for determining the status of an individual, comprising: obtaining a biological specimen from an individual, and assessing the activation state of a PI3K and/or mTOR pathway activatable element. In some embodiments, the PI3K pathway activatable element is PI3K, p110 isoforms, PDK-1, Akt (also referred to as protein kinase B or PKB) isoforms, PRAS40, Mdm2, TSC2, GSK3β, BAD, FOXO transcription factors, NFkB, mTOR, p70S6 kinase, Ribosomal S6, 4EBP1, Paxillin, PKCα, PKCβ, SGK, TSC1, Rictor or Raptor.

In some embodiments, the invention is a method for classifying a cell comprising: contacting the cell with a PI3K inhibitor, mTOR inhibitor, inhibitor of a PI3K and/or mTOR pathway protein or an inhibitor to more than one of these targets; determining the presence or absence of a change in activation level of an activatable element in the cell, and classifying the cell based on the presence or absence of the change in the activation level of the activatable element. In some embodiments, the change in activation level of the activatable element is an increase in activation level of the activatable element. In some embodiments, the cell is a cancer cell or a hematopoietic cell. In some embodiments, the presence or absence of a change in the activation level of the activatable element is compared to a normal cell contacted with the PI3K and/or mTOR inhibitor. In some embodiments, the presence or absence of a change in the activation levels of the activatable element is determined in the determining step. In some embodiments, the classification comprises classifying the cell as a cell that is correlated with a clinical outcome. In some embodiments, the clinical outcome is the presence or absence of a neoplastic, diabetic, or cancer condition. In some embodiments, the clinical outcome is the staging or grading of a neoplastic condition. In some embodiments, the classification further comprises determining a method of treatment. In some embodiments, the cell is subjected to a modulator, such as a cancer cell or hematopoietic cell modulator.

In some embodiments, the invention provides a method of determining the presence or absence of a condition in an individual comprising: subjecting a cell from the individual to a PI3K and/or mTOR pathway inhibitor; determining the activation level of an activatable element in the cell; and determining the presence or absence of the condition based on the activation level.

In some embodiments, the invention provides a method of correlating and/or classifying an activatable state of a cancer cell with a clinical outcome in an individual comprising: subjecting the cancer cell from the individual to a PI3K and/or mTOR pathway modulator; determining the activation level of an activatable element; and identifying a pattern of the activation level of the activatable element to determine the presence or absence of an alteration in signaling, wherein the presence of the alteration is indicative of a clinical outcome.

In some embodiments, the invention provides a method of analyzing the effect of a compound comprising: contacting a cell of interest with a compound of interest and analyzing activity of a PI3K and/or mTOR pathway protein in said cell.

### BRIEF DESCRIPTION OF THE DRAWINGS

FIG. 1 shows pathways downstream of PI3K and Akt.

FIG. 2 shows elected nodes that can determine the extent of PI3 kinase inhibition pathway nodes. In the presence of a Pan Class I inhibitor is shown selected nodes along pPRAS40^{T346}, the PI3 kinase pathway including: pBAD^{S136}, pAktS⁴⁷³⁵, and pS6^{S235/236}.

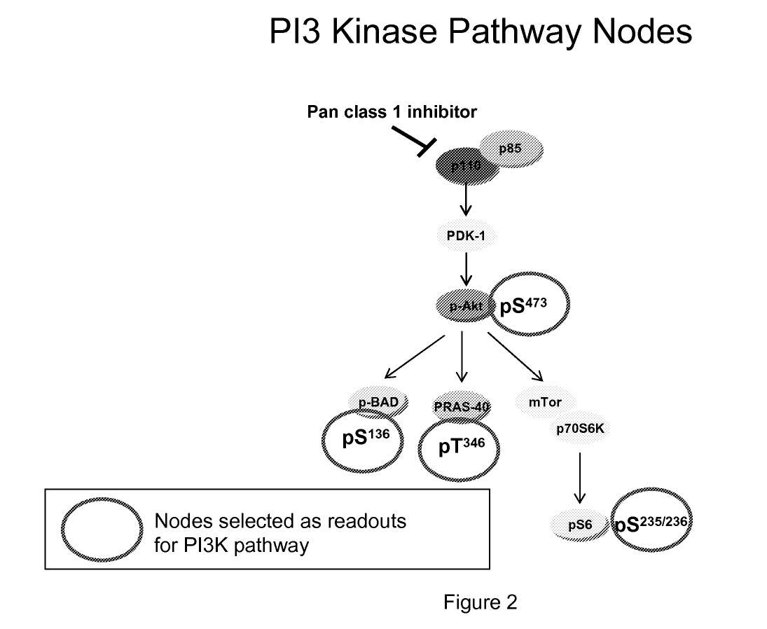

FIG. 3 shows how to distinguish mTOR kinase inhibition from PI3 kinase inhibition.

FIG. 4 shows subunits of TORC1 and TORC2 complexes and depicts the roles of both complexes in signaling pathways.

FIG. 5 shows predicted changes in phosphorylation states of signaling molecules contacted with mTOR inhibitors.

FIG. 6 shows one embodiment of a method that may elucidate any distinct functional consequences of mTOR inhibition

### DETAILED DESCRIPTION OF THE INVENTION

The present invention incorporates information disclosed in other applications and texts. The following publications are hereby incorporated by reference in their entireties: Haskell et al, Cancer Treatment, 5th Ed., W.B. Saunders and Co., 2001; Alberts et al., Molecular Biology of the Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and 1B, Growth Factors, 1996; and Immunophenotyping, Chapter 9: Use of Multiparameter Flow Cytometry and Immunophenotyping for the Diagnosis and Classfication of Acute Myeloid Leukemia, Stelzer, et al., Wiley, 2000.

Patents and applications that are also incorporated by reference include U.S. Pat. Nos. 7,381,535 and 7,393,656 and U.S. patent application Ser. Nos. 10/193,462; 11/655,785; 11/655,789; 11/655,821; 11/338,957, 61/048,886; 61/048,920; and 61/048,657.

Some commercial reagents, protocols, software and instruments that are useful in some embodiments of the present invention are available at the Becton Dickinson Website http://www.bdbiosciences.com/features/products/, and the Beckman Coulter website, http://www.beckmancoulter.com/Default.asp?bhfv=7.

Relevant articles include: High-content single-cell drug screening with phosphospecific flow cytometry, Krutzik et al., Nature Chemical Biology, 23: 132-42, December 2007; Irish et al., FLt3 ligand Y591 duplication and Bcl-2 over expression are detected in acute myeloid leukemia cells with high levels of phosphorylated wild-type p53, NeoplasiaBlood 109: 2589-96, 2007; Irish et al. Mapping normal and cancer cell signaling networks: towards single-cell proteomics, Nature Rev. Cancer, Vol. 6: 146155, 2006; and Irish et al., Single cell profiling of potentiated phospho-protein networks in cancer cells, Cell, Vol. 118, 1-20 Jul. 23, 2004; Schulz, K. R., et al., Single-cell phospho-protein analysis by flow cytometry, Curr Protoc Immunol, 2007, 78:8 Chapter 8: Units 8.17.1-20, 2007; Krutzik, P. O., et al., Coordinate analysis of murine immune cell surface markers and intracellular phosphoproteins by flow cytometry, J Immunol. 2005 Aug. 15; 175(4): 2357-65; Krutzik, P. O., et al., Characterization of the murine immunological signaling network with phosphospecific flow cytometry, J Immunol. 2005 Aug. 15; 175(4): 2366-73, 2005; Shulz et al., Current Protocols in Immunology 2007, 78:8.17.1-20; Stelzer et al. Use of Multiparameter Flow Cytometry and Immunophenotyping for the Diagnosis and Classfication of Acute Myeloid Leukemia, Immunophenotyping, Wiley, 2000; and Krutzik, P. O. and Nolan, G. P., Intracellular phospho-protein staining techniques for flow cytometry: monitoring single cell signaling events, Cytometry A. 2003 October; 55(2):61-70, 2005; Hanahan D., Weinberg, The Hallmarks of Cancer, CELLCell 100: 2000 Jan. 7; 100(1) 57-70, 2000; Krutzik et al, High content single cell drug screening with phophosphospecific flow cytometry, Nat Chem Biol. 2008 February; 4(2):132-42, 2008; Gewinner, C., et al. Evidence that Inositol Polyphosphate 4-Phosphatase Type II Is a Tumor Suppressor that Inhibits PI3K Signaling. Cancer Cell (2009) 16: 115-25; Foukas et al. (2006) Nature 44:366-70; Knight et al. (2006) Cell 125:733-47; Yuan and Cantley (2008) Oncogene 27:5497-5510; Liu et al. (2009) Nature Reviews Drug Discovery 8:627-644; Taniguchi et al. (2006) PNAS 103:12093-7; Garcia-Echeverria and Sellers (2008) Oncogene 27:5511-26; Martin-Berenjeno and Vanhaesebroeck (2009) Cancer Cell 16:449-50; and Lee et al. (2007) Science 317:206-7. Experimental and process protocols and other helpful information can be found at http://proteomics.stanford.edu. The articles and other references cited below are also incorporated by reference in their entireties for all purposes.

The discussion below describes some of the preferred embodiments with respect to particular diseases. However, it should be appreciated that the principles may be useful for the analysis of many other diseases as well. Without being limited, example diseases include cancers, metabolic disorders and autoimmune diseases. Example, cancers include glioblastoma, colon, breast, thyroid, ovarian, prostate, lung, melanoma and pancreatic cancers. See Hanahan D., Weinberg, The Hallmarks of Cancer, Cell, Jan. 7, 2000, 100(1): 57-70 cited above. Other examples are shown in Wood et al, The Genomic Landscapes of Human Breast and Colorectal Cancers. Science 318: 1108-1113, 2007; Jones et al., Core Signaling Pathways in Human Pancreatic Cancers Revealed by Global Genomic Analyses. Science 321: 1801-1806, 2008; and Parsons et al., An Intergrated Genomic Analysis of Human Glioblastoma Multiforme. Science 321: 1807-1812, 2008, which are all incorporated by reference in their entireties.

In some embodiments, the method provides for a diagnosis of a condition, such as cancer, an immune disorder or diabetes. In other embodiments, the method provides for the classification of a condition, such as cancer. Classification can include, but is not limited to, a determination of disease subtype, disease stage, disease activity level, and responsiveness to treatment. In other embodiments, a prediction is made as to the likelihood of a possible disease-related outcome. In one embodiment, the prediction relates to the future course of disease activity. The future course of predicted activity may be a change, as in an increase or decrease in disease activity, or the future course of activity may be predicted to remain substantially similar to the activity level at the time of analysis.

In some embodiments, a prediction is made as to the likelihood of responsiveness to one or more treatments. Treatments about which a prediction can be made can include a treatment the subject has not yet received, or changes in treatments with which a subject is being treated. Changes can include, but are not limited to changes in dose, dosing schedule, route of administration, and/or combination with additional treatments. In some embodiments, a prediction guides the selection of or changes in treatment received by a subject.

In some embodiments, the at least one modulator with which a cell from a subject is contacted is a therapeutic agent, wherein the therapeutic agent is one used to treat a condition, such as cancer.

In some embodiments, the invention is a method for determining the status of an individual, comprising: obtaining a biological specimen from an individual, and assessing the activation state of a PI3K and/or mTOR pathway activatable element. In some embodiments, the PI3K pathway activatable element is PI3K, p110 isoforms, PDK-1, Akt (also referred to as protein kinase B or PKB) isoforms, PRAS40, Mdm2, TSC2, GSK3β, BAD, FOXO transcription factors, NFkB, mTOR, p70S6 kinase, Ribosomal S6, 4EBP1, Paxillin, PKCα, PKCβ, SGK, TSC1, Rictor or Raptor.

In some embodiments, the invention is a method for classifying a cell comprising: contacting the cell with a PI3K inhibitor, mTOR inhibitor, inhibitor of a PI3K and/or mTOR pathway protein or an inhibitor to more than one of these targets; determining the presence or absence of a change in activation level of an activatable element in the cell, and classifying the cell based on the presence or absence of the change in the activation level of the activatable element. In some embodiments, the change in activation level of the activatable element is an increase in activation level of the activatable element. In some embodiments, the cell is a cancer cell or a hematopoietic cell. In some embodiments, the presence or absence of a change in the activation level of the activatable element is compared to a normal cell contacted with the PI3K and/or mTOR inhibitor. In some embodiments, the presence or absence of a change in the activation levels of the activatable element is determined in the determining step. In some embodiments, the classification comprises classifying the cell as a cell that is correlated with a clinical outcome. In some embodiments, the clinical outcome is the presence or absence of a neoplastic, diabetic, or cancer condition. In some embodiments, the clinical outcome is the staging or grading of a neoplastic condition. In some embodiments, the classification further comprises determining a method of treatment. In some embodiments, the cell is subjected to a modulator, such as a cancer cell or hematopoietic cell modulator.

In some embodiments, the invention provides a method of determining the presence or absence of a condition in an individual comprising: subjecting a cell from the individual to a PI3k and/or mTOR pathway inhibitor; determining the activation level of an activatable element in the cell; and determining the presence or absence of the condition based on the activation level.

In some embodiments, the invention provides a method of correlating and/or classifying an activatable state of a cancer cell with a clinical outcome in an individual comprising: subjecting the cancer cell from the individual to a PI3K and/or mTOR pathway modulator; determining the activation level of an activatable element; and identifying a pattern of the activation level of the activatable element to determine the presence or absence of an alteration in signaling, wherein the presence of the alteration is indicative of a clinical outcome.

In some embodiments, the invention provides a method of analyzing the effect of a compound comprising: contacting a cell of interest with a compound of interest and analyzing activity of a PI3K and/or mTOR pathway protein in said cell.

One embodiment of the invention measures multiple mechanisms by which the PI3 kinase (PI3K) and/or mammalian target of rapamycin (mTOR) pathways may be activated. The mechanism by which a pathway is activated can impact several health care issues, such as drug development, therapeutic treatments, patient management, or diagnosis, and also the analysis of how a cell, such as a tumor cell, may change under therapeutic pressure. One embodiment of the invention consists of the use of biological assays, including but not limited to multiparameter flow cytometry, to measure PI3K-dependent and PI3K-independent signaling simultaneously in single cells from a heterogeneous population. Once the mechanism of pathway activation has been identified, a researcher will be able to select methods to inhibit its activity. For example, the process may involve measuring the levels of phosphorylated a protein, for example p-Akt, comparing the amount of activated protein to overall protein level, and then taking an action, such as selecting a particular inhibitor, adjusting dosing, schedule, etc. Protein modifications, including but not limited to phosphorylation, can serve as measurements of signaling pathway activity. Thus, the methods of the invention can be used to determine whether the inhibitor affects one or more components of the PI3 kinase pathway.

One embodiment of the present invention discloses ways of using phosphoflow to assist in the development of PI3 kinase isoform specific inhibitors, either as single agents or in combination with other targeted therapies. One method that will be useful is multiparametric phosphoflow technology which can simultaneously measure activity of multiple pathways at the single cell level within heterogeneous cell populations. Other methods which allow the researcher to examine multiple signaling pathways will also be useful.

One embodiment of the invention involves methods for monitoring response of neoplasias to drugs specifically designed to correct the molecular abnormalities. Some methods can be useful to select dose/scheduling of these drugs in these patients.

These methods can then be employed to create test-specific assays and kits to determine patient response to drugs that target p110 isoforms, PDK-1, or Akt isoforms.

One embodiment of the invention is an array or kit of test-specific reagents, which may include antibodies that can measure levels of expression and/or recognize one or more of the following:
a. p110 isoforms: epitopes within particular domains, or multiple domains in one or more conformation;
b. PDK-1: phosphorylated and/or non-phosphorylated epitopes within PDK-1;
c. Akt isoforms: phosphorylated and/or non-phosphorylated epitopes within Akt isoforms;
d. PRAS40: phosphorylated and/or non-phosphorylated epitopes within PRAS40;
e. Mdm2: phosphorylated and/or non-phosphorylated epitopes within Mdm2;
f. TSC2;
g. GSK3β: phosphorylated and/or non-phosphorylated epitopes within GSK-3β;
h. BAD: phosphorylated and/or non-phosphorylated epitopes within BAD;
i. FOXO transcription factors: phosphorylated and/or non-phosphorylated epitopes within FOXO transcription factors;
j. NFKB/p65/RelA: phosphorylated and/or non-phosphorylated epitopes within NFKB/p65/RelA;
k. mTOR: phosphorylated and/or non-phosphorylated epitopes within mTor;
l. p70S6 kinase: phosphorylated and/or non-phosphorylated epitopes within p70S6 kinase;
m. Ribosomal S6: phosphorylated and/or non-phosphorylated epitopes within Ribosomal S6;
n. 4EBP1: phosphorylated and/or non-phosphorylated epitopes within 4EBP1;
o. Paxillin: phosphorylated and/or non-phosphorylated epitopes within Paxillin;
p. PKCα: phosphorylated and/or non-phosphorylated epitopes within PKCα;
q. PKCβ: phosphorylated and/or non-phosphorylated epitopes within PKCβ;
r. SGK: phosphorylated and/or non-phosphorylated epitopes within SGK;
s. TSC1;
t. Raptor; and
u. Rictor.

### General Methods

The following will discuss research and diagnostic methods, instruments, reagents, kits, and the biology involved with PI3 kinase pathway and their inhibitors. One aspect of the invention involves subjecting a cell to at least one of a plurality of compounds; analyzing states or nodes using techniques known in the art, such as phosphoflow cytometry, where individual cells are simultaneously analyzed for multiple characteristics: activity of gain-of-function mutations, expression levels and activity of PI3 kinase pathway components, expression levels and activity of regulatory proteins, phosphorylation status, epigenetic changes, post-translational modifications of PI3 kinase pathway components, post translational modifications of regulatory proteins, microRNA changes, and activity and expression; correlating the results of the analysis with a response to a compound; and classifying said cells into clinical outcomes.

In some embodiments, the present invention is directed to select at least one of a plurality of compounds for optimization and preclinical studies. In some embodiments, the present invention is directed to determining dosing and scheduling of at least one of a plurality of compounds that may be used as therapeutics. In some embodiments, the invention employs techniques, such as flow cytometry, imaging approaches, mass spec based flow cytometry, nucleic acid microarrays, quantitative PCR or reverse-transcriptase PCR, or other phenotypic assays.

In some embodiments, the invention is directed to methods for determining the activation level of one or more activatable elements in a cell upon treatment with one or more modulators. The activation of an activatable element in the cell upon treatment with one or more modulators can reveal operative pathways in a condition that can then be used, e.g., as an indicator to predict course of the condition, identify risk group, predict an increased risk of developing secondary complications, choose a therapy for an individual, predict response to a therapy for an individual, determine the efficacy of a therapy in an individual, and determine the clinical outcome for an individual.

In some embodiments, the invention is directed to methods of determining a phenotypic profile of a population of cells by exposing the population of cells to a plurality of modulators in separate cultures, wherein at least one of the modulators is an inhibitor, determining the presence or absence of an increase in activation level of an activatable element in the cell population from each of the separate culture and classifying the cell population based on the presence or absence of the increase in the activation of the activatable element from each of the separate culture.

One or more cells or cell types, or samples containing one or more cells or cell types, can be isolated from body samples. The cells can be separated from body samples by centrifugation, elutriation, density gradient separation, apheresis, affinity selection, panning, FACS, centrifugation with Hypaque, etc. By using antibodies specific for markers identified with particular cell types, a relatively homogeneous population of cells may be obtained. Alternatively, a heterogeneous cell population can be used. Cells can also be separated by using filters. For example, whole blood can also be applied to filters that are engineered to contain pore sizes that select for the desired cell type or class. Rare pathogenic cells can be filtered out of diluted, whole blood following the lysis of red blood cells by using filters with pore sizes between 5 to 10 μm, as disclosed in U.S. patent application Ser. No. 09/790,673. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Patent Application Nos. 61/048,886; 61/048,920; and 61/048,657. Exemplary established cell lines may also be used, such as (for hematological tumors) U937, THP, Kg-1, OPM2, MM1, and ESM; (for solid tumors) U87Mg, PC3, BT474, and A549. See also, the commercial products from companies such as BD and BCI as identified above.

See also U.S. Pat. Nos. 7,381,535 and 7,393,656. All of the above patents and applications are incorporated by reference as stated above.

The term “patient” or “individual” as used herein includes humans as well as other mammals. The methods generally involve determining the status of an activatable element. The methods also involve determining the status of a plurality of activatable elements.

The analysis of a cell and the determination of the status of an activatable element can comprise classifying a cell as a cell that is correlated to a patient response to a treatment. In some embodiments, the patient response is selected from the group consisting of complete response, partial response, nodular partial response, no response, progressive disease, stable disease and adverse reaction.

The classification of a rare cell according to the status of an activatable element can comprise classifying the cell as a cell that can be correlated with minimal residual disease or emerging resistance. See U.S. Application No. 61/048,886, which is incorporated by reference.

The classification of a cell according to the status of an activatable element can comprise selecting a method of treatment. Examples of treatment methods include, but are not limited to, compounds that control some of the symptoms, such as aspirin and antihistamines, compounds that stimulate red blood cell production, such as erythropoietin or darbepoietin, compounds that reduce platelet production, such as hydroxyurea, anagrelide, and interferon-alpha, compounds that increase white blood cell production, such as G-CSF, chemotherapy, biological therapy, radiation therapy, phlebotomy, blood cell transfusion, bone marrow transplantation, peripheral stem cell transplantation, umbilical cord blood transplantation, autologous stem cell transplantation, allogeneic stem cell transplantation, syngeneic stem cell transplantation, surgery, induction therapy, maintenance therapy, and other therapy.

In some embodiments, cells (e.g. normal cells) other than the cells associated with a condition (e.g. cancer cells) or a combination of cells are used, e.g., in assigning a risk group, predicting an increased risk of relapse, predicting an increased risk of developing secondary complications, choosing a therapy for an individual, predicting response to a therapy for an individual, determining the efficacy of a therapy in an individual, and/or determining the prognosis for an individual. For example, in the case of cancer, infiltrating immune cells might determine the outcome of the disease. Alternatively, a combination of information from the cancer cell plus the immune cells in the blood that are responding to the disease, or reacting to the disease can be used for diagnosis or prognosis of the cancer.

In some embodiments, the analysis involves looking at multiple characteristics of the cell in parallel after contact with the compound. For example, the analysis can examine drug transporter function; drug transporter expression; drug metabolism; drug activation; cellular redox potential; signaling pathways; DNA damage repair; and apoptosis. Analysis can assess the ability of the cell to undergo the process of apoptosis after exposure to the experimental drug in an in vitro assay as well as how quickly the drug is exported out of the cell or metabolized.

In some embodiments, the methods of the invention provide methods for classifying a cell population or determining the presence or absence of a condition in an individual by subjecting a cell from the individual to a modulator and an inhibitor, determining the activation level of an activatable element in the cell, and determining the presence or absence of a condition based on the activation level. In some embodiments, the activation level of a plurality of activatable elements in the cell is determined. The inhibitor can be an inhibitor as described herein. In some embodiments, the inhibitor is a phosphatase inhibitor. In some embodiments, the inhibitor is H₂O₂. The modulator can be any modulator described herein. In some embodiments, the methods of the invention provides for methods for classifying a cell population by exposing the cell population to a plurality of modulators in separate cultures and determining the status of an activatable element in the cell population. In some embodiments, the status of a plurality of activatable elements in the cell population is determined. In some embodiments, at least one of the modulators of the plurality of modulators is an inhibitor. The modulator can be at least one of the modulators described herein. In some embodiments, at least one modulator is selected from the group consisting of SDF-1α, IFN-α, IFN-γ, IL-10, IL-6, IL-27, G-CSF, FLT-3L, M-CSF, SCF, PMA, Thapsigargin, H₂O₂, etoposide, AraC, daunorubicin, staurosporine, and benzyloxycarbonyl-Val-Ala-Asp (OMe) fluoromethylketone (ZVAD), IL-3, IL-4, GM-CSF, EPO, LPS, TNF-α, and CD40L, and a combination thereof. The above listed modulators are useful, among other things, in hematopoietic cells for use in monitoring hematological disorders or as surrogate markers for non-hematological disorders (e.g. solid tumors). Other modulators can also be used such as EGF family ligands, PDGF family ligands, FGF family ligands, VEGF family ligands, Ang1, Ang2, HGF and IGF1. The above listed modulators are useful, among other things, as markers on tumor cells of epithelial origin.

In some embodiments of the invention, the status of an activatable element is determined by contacting the cell population with a binding element that is specific for an activation state of the activatable element. In some embodiments, the status of a plurality of activatable elements is determined by contacting the cell population with one or more of a plurality of binding elements. In some embodiment, each binding element is specific for an activation state of an activatable element.

In some embodiments, the methods of the invention provide methods for determining a phenotypic profile of a population of cells by exposing the population of cells to a plurality of modulators (recited herein) in separate cultures, wherein at least one of the modulators is an inhibitor, determining the presence or absence of an increase in activation level of an activatable element in the cell population from each of the separate cultures and classifying the cell population based on the presence or absence of the increase in the activation of the activatable element from each of the separate culture. Patterns and profiles of one or more activatable elements are detected using the methods known in the art including those described herein. In some embodiments, patterns and profiles of activatable elements that are cellular components of a cellular pathway or a signaling pathway are detected using the methods described herein. For example, patterns and profiles of one or more phosphorylated polypeptides are detected using methods known in art including those described herein.

In some embodiments, the invention provides methods to carry out multiparameter flow cytometry for monitoring phospho-protein responses to various factors in myeloproliferative cancers at the single cell level. Phospho-protein members of signaling cascades and the kinases and phosphatases that interact with them are required to initiate and regulate proliferative signals in cells. Apart from the basal level of protein phosphorylation alone, the effect of potential drug molecules on these network pathways was studied to discern unique cancer network profiles, which correlate with the genetics and disease outcome. Single cell measurements of phospho-protein responses reveal shifts in the signaling potential of a phospho-protein network, enabling categorization of cell network phenotypes by multidimensional molecular profiles of signaling. See U.S. Pat. No. 7,393,656. See also IRISH et. al., Single cell profiling of potentiated phospho-protein networks in cancer cells. Cell. 2004, vol. 118, p. 1-20.

Flow cytometry is useful in a clinical setting, since relatively small sample sizes, as few as 10,000 cells, can produce a considerable amount of statistically tractable multidimensional signaling data and reveal key cell subsets that are responsible for a phenotype. See U.S. Pat. Nos. 7,381,535 and 7,393,656, and also Krutzik et al., 2004).

### Disease Conditions

The methods of the invention are applicable to any condition in an individual involving, indicated by, and/or arising from, in whole or in part, altered physiological status in a cell. In some embodiments of the present invention, the altered physiological state is an alteration in one or more PI3K and/or mTOR pathway proteins. The term “physiological status” includes mechanical, physical, and biochemical functions in a cell. In some embodiments, the physiological status of a cell is determined by measuring characteristics of cellular components of a cellular pathway. Cellular pathways are well known in the art. In some embodiments the cellular pathway is a signaling pathway. Signaling pathways are also well known in the art (see, e.g., Hunter T., Cell 100(1): 113-27 (2000); Cell Signaling Technology, Inc., 2002 Catalogue, Pathway Diagrams pgs. 232-253). A condition involving or characterized by altered physiological status may be readily identified, for example, by determining the state in a cell of one or more activatable elements, as taught herein. See U.S. Ser. No. 61/120,320.

In some embodiments, the present invention is directed to methods for analyzing the effects of a compound on the PI3 kinase pathway in one or more cells in a sample derived from an individual having or suspected of having a condition, such as cancer. For example, conditions include any solid or hematological cancer. Other examples include immune disorders, including autoimmune disorders, muscular sclerosis (MS), arthritis, allergic encephalomyelitis, and other immunosuppressive-related disorders, metabolic disorders (e.g., diabetes), reducing intimal thickening following vascular injury, and misfolded protein disorders (e.g., Alzheimer's Disease, Gaucher's Disease, Parkinson's Disease, Huntington's Disease, cystic fibrosis, macular degeneration, retinitis pigmentosa, and prion disorders), hamartoma syndromes, such as tuberous sclerosis and Cowden Disease (also termed Cowden syndrome and multiple hamartoma syndrome), and genetic muscle disorders and myopathies, such as human myotubular myopathy, cardiovascular, viral and other disease conditions. In some embodiments, the invention allows for identification of prognostically and therapeutically relevant subgroups of the conditions and prediction of the clinical course of an individual. Cell lines may also be used for testing.

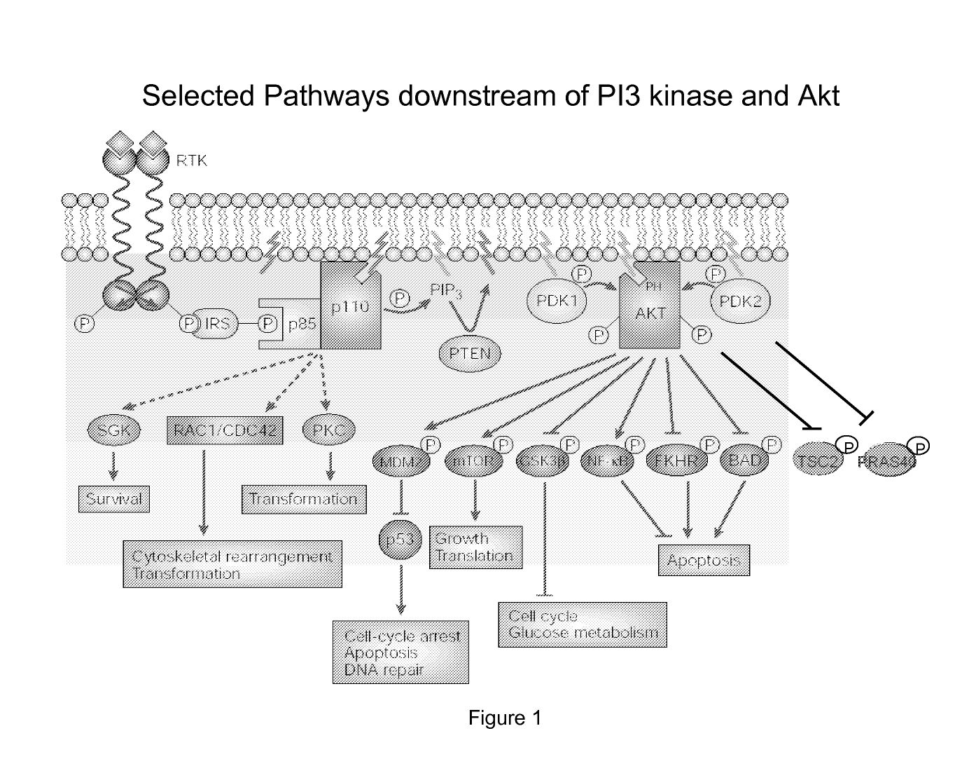

Phosphatidylinositol 3-kinases (PI3K) are ubiquitously expressed lipid kinases that phosphorylate phosphoinositides at the D-3 position of the inositol ring. The products of PI3K-catalysed reactions, phosphatidylinositol 3,4,5-trisphosphate, phosphatidylinositol 3,4 bisphosphate and phosphatidylinositol 3-phosphate are second messengers whose levels are tightly regulated by phosphatases such as Phosphatase and TENsin homologue (PTEN) that acts in an opposing role to remove the D-3 phosphate. The lipid products of PI3Ks bind or associate with pleckstrin homology containing proteins, including but not limited to the Akt serine/threonine kinases. This kinase phosphorylates a broad range of protein targets (see, for example, FIG. 1) with important consequences for a number of cellular processes, including but not limited to, proliferation, survival, metabolism, differentiation and motility. These protein target nodes are also referred to here as PI3K pathway proteins. Examples of PI3K and/or mTOR pathway proteins include, but are not limited to, p110 isoforms, PDK-1, Akt isoforms, PRAS40, Mdm2, TSC2, GSK3β, BAD, FOXO transcription factors, NFkB, mTOR, p70S6 kinase, Ribosomal S6, 4EBP1, Paxillin, PKCα, PKCβ, SGK, TSC1, pBAD^{S136}, pAkt^{S473}, ppRA^{S40T346} pAkt^{S3085} ps6^{S235/2365} Rictor and Raptor.

The phosphatidylinositol-3-kinase family is composed of Class 1, Class II and Class III complexes. Class I PI3Ks are heterodimeric molecules composed of a regulatory and a catalytic subunit; they are further divided between IA and IB subsets on sequence similarity. The Class IA PI3K subgroup consists of three catalytic subunits, p110α, β or δ, that form heterodimers with one of five regulatory subunits; p85α, p55α, p50α, p85β or p55γ. Class 1A PI3Ks are activated in response to many external modulators, including but not limited to, growth factors, integrins, chemokines, and cytokines The first two p110 isoforms (α and β) are expressed in all cells, but p110δ is primarily expressed in leukocytes and it has been suggested it evolved in parallel with the adaptive immune system. The class 1B PI3K consists of one member, a heterodimer of a catalytic p110γ and a regulatory subunit which comprises p101 or p84, and is activated by G-protein coupled receptors (see Stephens, L. et al. Phosphoinositide 3-kinases as drug targets in cancer. Curr. Opin. Pharmacology (2005) δ: 357-65).

PI3Ks have been linked to an extraordinarily diverse group of cellular functions, including cell growth, proliferation, differentiation, motility, signal transduction, survival and intracellular trafficking. Many of these functions relate to the ability of class I PI3Ks to activate Akt. The class IA PI3K p110α is mutated in many cancers (Velasco et al. (2006) Hum Pathol 37:1465-72). Many of these mutations cause the kinase to be more active. The Ptdlns(3,4,5)P3 phosphatase PTEN which antagonizes PI3K signaling is absent from many tumors. Hence, PI3K activity can contribute to cellular transformation and the development of cancer.

The PI3K pathway was first linked to cancer by the finding that the avian sarcoma virus 16 genome encodes an oncogene derived from the cellular PI3K gene. Subsequently, researchers have observed that each of the major components of the PI3K pathway is frequently mutated or overexpressed in a broad range of human cancers (Yuan, T. L. and Cantley, L. C. PI3K pathway alteration in cancer: variations on a theme. Oncogene 27: 5497-5510, 2008). These major components of the PI3K pathway include, but are not limited to, receptor tyrosine kinases (RTKs; for example EGFR and HER2), PTEN, Akt, and the p110α subunit of PI3K. In healthy cells, ligand binding induces RTKs to activate PI3 kinase (PI3K), which phosphorylates the 3′ position of the inositol ring of phosphatidyl inositol 4-phosphate, or phosphatidyl inositol 4,5 phosphate to generate, respectively, the inositol lipid second messengers phosphatidylinositol 3,4 bisphosphate (PIP2), and phosphatidylinositol 3,4,5 bisphosphate (PIP3). These second messengers bind to the Pleckstrin Homology (PH) domains of PDK1 and Akt to recruit them to the plasma membrane, resulting in their subsequent activation through phosphorylation (for review, see Katso, R., et al. Cellular function of phosphoinositide 3-kinases: implications for development, homeostasis, and cancer. Annu Rev Cell Dev Biol. (2001)17: 615-75.

The p110α protein is the catalytic subunit of PI3K, and is encoded by PIK3CA. The kinase and helical domain-encoding portions of PIK3CA contain oncogenic missense mutations in up to 27% of breast, endometrial, colorectal, urinary tract, and ovarian cancers (Samuels et al. High frequency of mutations of the PIK3CA gene in human cancers. Science 304: 554, 2004). These mutations most often occur at the hotspot codons E542, E545, and H1047, and have been shown to confer constitutive kinase activity (Samuels et al. Mutant PIK3CA promotes cell growth and invasion of human cancer cells. Cancer Cell 7: 561-73, 2005). Furthermore, PIK3CA is frequently amplified in various human cancers. (Engelman, J. A., et al. The evolution of phosphatidylinositol 3-kinases as regulators of growth and metabolism. Nat Rev Genet 7: 606-19, 2006). PTEN negatively regulates PI3K signaling in healthy cells by dephosphorylating the second messenger, PIPS. PTEN has been identified as a tumor suppressor, and mutations that inactivate PTEN are found in various cancers (Salmena, L. et al. Tenets of PTEN Tumor Suppression. Cell 133: 403-14. 2008). Cancer cells contain PIK3CA and PTEN mutations more frequently than would be predict by chance alone, suggesting that PIK3CA gain-of-function and PTEN loss-of-function mutations are not entirely redundant in oncogenesis (Yuan, T. L. and Cantley, L. C. Oncogene 27: 5497-5510, 2008). In contrast to PIK3CA, no cancer-associated somatic mutations have been found to date in PIK3CB or PIK3CD, which encode the p110β and p110γ isoforms respectively, although overexpression of these genes in cell lines suggests that they might have tumorigenic potential. Consistent with this latter observation, increased levels of p110β and p110δ proteins have been found in a variety of cancers. Other genetic alterations within the PI3K pathway have also been also identified, including mutations in p85.

There are other clues that the different PI3K pathway components may function in different mechanisms of oncogenesis. In a mouse model of cancer generated by PTEN ablation, conditional knockout of PIK3CB, but not PIK3CA impeded tumorigenesis (Jia, S. et al. Essential roles of PI(3)K-p110beta in cell growth, metabolism and tumorigenesis. Nature 454: 776-79, 2008.). Furthermore, gain-of-function mutations in PIK3CA may mediate oncogenesis through Akt-dependent and Akt-independent mechanisms (Vasudevan, K. M., et al, Akt-independent signaling downstram of oncogenic PIK3CA mutataions in human cancer. Cancer Cell 16: 21-32, 2009). In some embodiments, the methods of the present invention can measure gain-of-function mutations in a PI3K pathway protein. In some embodiments, methods of the present invention can measure loss-of-function mutations in a PI3K pathway protein. In some PIK3CA-mutant cancer cell lines, P110α does not induce Akt activation, but does induce PDK1 activation and recruitment to the cell membran. In these cell lines, PDK1 mediates SGK3 activation, which is required for survival of these cancer cells (Vasudevan, K. M., et al. Cancer Cell 16: 21-32, 2009). Thus, Akt signaling and SGK3 signaling may represent alternate mechanisms of PI3K-mediated oncogenesis.

Multiple studies have indicated that oncogenic alterations in PI3K signaling are not functionally equivalent, and do not necessarily just result in linear changes in signaling activity (Vasudevan, K. M., et al. Cancer Cell 16: 21-32, 2009; Yuan, T. L. and Cantley, L. C. Oncogene 27: 5497-5510, 2008). Instead, alterations in various nodes in the PI3K network may affect non-linear signaling, for example via negative feedback loops, crosstalk from other pathways, or activation of non-overlapping pathways. As an additional argument against complete functional redundancy, some mutations frequently coexist in the same tumor cell, for example PI3KCA gain-of-function and PTEN loss-of-function. There would be no co-selection for these mutations it they were functionally redundant. Instead, coexistence of two or more mutations in the pathway in the same tumor suggests selection for two or more different but synergistic mechanisms, neither of which is alone sufficient to confer oncogenecity. On the other hand, RAS mutations appear to be mutually exclusive with PIK3CA, suggesting the combination of both signaling mechanisms may be disadvantageous for cancer cells (Yuan, T. L. and Cantley, L. C. Oncogene 27: 5497-5510, 2008). Thus, the genetic background of a tumor may have important effects on the mechanism of PI3K activation. In some embodiments, the methods of the present invention include methods of measuring more than one PI3K and/or mTOR pathway protein simultaneously. The mutations can distinguish between different conditions and determine different methods of treatment to pursue.

The genetic evidence that different mechanisms of altered PI3K signaling can lead to cancer has implications for small molecule drug development. For example, the selection of which p110 isoform or isoforms to target depends on the mechanism of PI3K pathway disruption, if any, in cancer cells. Therapeutic efficacy may be improved by the selection of an appropriate targeted therapeutic or combination of therapeutics appropriate for a specific genetic background.

The p110α, β, δ and γ isoforms regulate different aspects of immune disorders. Immune disorders include inflammatory diseases, autoimmune diseases, organ and bone marrow transplant rejection and other disorders associated with T cell-mediated immune response or mast cell-mediated immune response. Non-limiting examples of immune disorders include acute or chronic inflammation, an allergy, contact dermatitis, psoriasis, rheumatoid arthritis, multiple sclerosis, type 1 diabetes, inflammatory bowel disease, Guillain-Barre syndrome, Crohn's disease, ulcerative colitis, cancer, graft versus host disease (and other forms of organ or bone marrow transplant rejection), autoimmune hemolytic anemia, autoimmune hepatitis, Berger's disease or IgA nephropathy, Celiac Sprue, chronic fatigue syndrome, dermatomyositis, fibromyalgia, Grave's disease, Hashimoto's thyroiditis, idiopathic thrombocytopenia purpura, lichen planus, multiple sclerosis, myasthenia gravis, rheumatic fever, scleroderma, Sjorgren syndrome, systemic lupus erythematosus, and vitiligo.

PI3Ks are also a key component of the insulin signaling pathway. Thus, PI3K signaling can be involved in diabetes, such as in Diabetes mellitus. The p110α and β associated lipid kinase activity has been demonstrated in insulinoma cells. PI3 kinase inhibition with reagents such as wortmannin and LY294002 enhances glucose-dependent insulin secretion. Activity of p110γ has been demonstrated in insulinoma cells, while protein expression has been shown in human, dog, rat and mouse pancreas by immunohistochemistry.

mTOR (mammalian target of rapamycin) is a serine/threonine kinase originally identified as TOR in yeast (Saccharomyces cerevisiae), and discovered during a screen for resistance to the immunosuppressant rapamycin (also known by its USAN generic name, sirolimus) (see, e.g., Kunz et al. (1993) Cell (73): 585, or U.S. Pat. No. 3,929,992). It is a member of the PI3K (phosphoinositide 3-kinases) family of protein kinases, identified by homology within its catalytic domain.

In yeast and mammals, the identification of two structurally and functionally distinct multiprotein TOR complexes (TORC1 and TORC2) has provided a molecular basis for the complexity of TOR signaling. mTOR activity is regulated by at least three upstream inputs: amino acids, glucose, and growth factors.

One embodiment of the present invention identifies multiple roles of the mTOR kinase in both normal and aberrantly regulated cellular conditions. Identification of these roles of the mTOR kinase can assist important health care decisions, for example drug development strategy, selection and dosing of therapeutic treatments, patient management, diagnosis, and prognosis. One embodiment of the invention uses biological assays, including but not limited to, multiparameter flow cytometry, to measure mTOR-dependent and mTOR-independent signaling simultaneously in single cells from a heterogeneous population. These single cell measurements may be analyzed and/or complied into profiles that may correlate with any normal or abnormal cellular condition as further described herein. The profiles may have diagnostic and/or prognostic utility and may be referred to as single cell network profiles (SCNP).

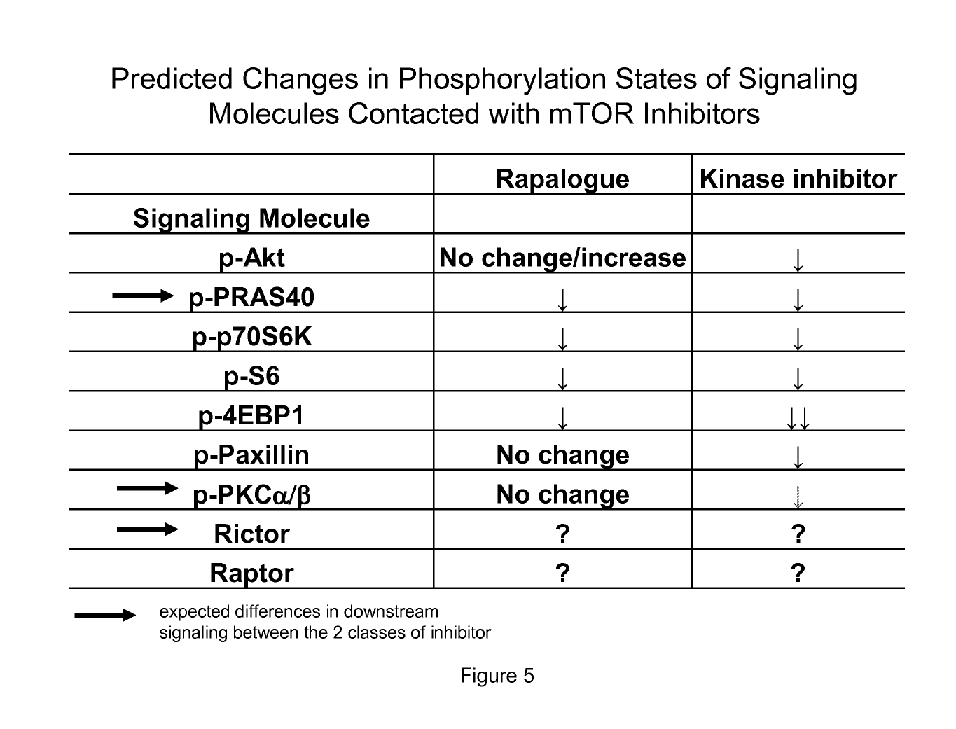

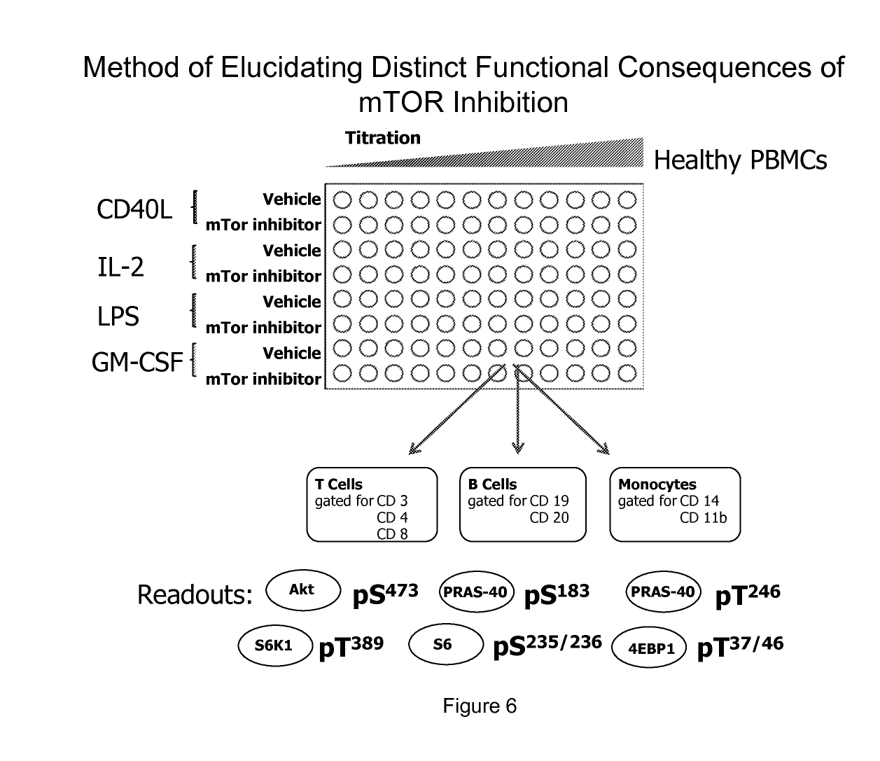

For example, distinct and/or partially overlapping substrate specificities of the mTOR containing multiprotein complexes TORC1 and TORC2 may be determined by contacting a single cell or a population of cells with allosteric and/or kinase inhibitors of mTOR. Currently available allosteric mTOR inhibitors inhibit only TORC1, whereas kinase inhibitors inhibit both TORC1 and TORC2. Single cell types, such as a cell line, or populations of cells within a complex biological sample including but not limited to peripheral blood mononuclear cells or bone marrow mononuclear cells, may be treated with an allosteric inhibitor alone, a kinase inhibitor alone, or both inhibitors simultaneously or sequentially. The allosteric mTOR inhibitors described below may change the phosphorylation state of downstream proteins within an mTOR pathway in a manner distinct from any phosphorylation state change elicited by the mTOR kinase inhibitors, also described below. The allosteric inhibitor rapamycin may decrease p-p70S6K phosphorylation and not p-paxillin phosphorylation, while the kinase inhibitor torinl may decrease p70S6K phosphorylation, p-Akt and p-paxillin phosphorylation. See FIG. 5. See also FIG. 6 for an illustration of an experiment designed to elucidate any distinct functional consequences of mTOR inhibition. The allosteric and kinase mTOR inhibitors may produce distinct SCNPs that may then inform may health care decisions.

As another example, distinct and/or partially overlapping substrate specificities of the PI3K and mTOR proteins, or any other PI3K and/or mTOR pathway proteins may be determined by contacting a single cell or a population of cells with a modulator, such as allosteric and/or kinase inhibitors of PI3K, mTOR, or any other PI3K and/or mTOR pathway protein.

In some embodiments, the invention can be used to distinguish modulators that are specific for the PI3K pathway, mTOR pathway, or modulate both the PI3K and mTOR pathway. In some embodiments, the invention can also be used to distinguish modulators that are specific for the above pathways and characterize side effects associated with the modulator, or each of the above stated pathways. In some embodiments, characterization of side effects associated with particular modulators, the PI3K pathway or mTOR pathway can be used to determine uses of particular modulators in treatment of an individual.

The invention contemplates use of one or more PI3K and/or mTOR pathway protein for use in the methods of the present invention. For example, other PI3K and/or mTOR pathway proteins include Akt, PDK1, serum and glucocorticoid-regulated kinase (SGK), ataxia telangiectasia mutated (ATM), ataxia telangiectasia, Rad3 related (ATR), and DNA-dependent protein kinase (DNA-PK).

One embodiment of the invention identifies multiple mechanisms of PI3 kinase pathway activation. Identification of the mechanism of pathway activation can assist important health care decisions, for example drug development strategy, selection and dosing of therapeutic treatments, patient management, diagnosis, and prognosis. One embodiment of the invention consists of the use of biological assays, including but not limited to, multiparameter flow cytometry, to measure PI3K-dependent and PI3K-independent signaling simultaneously in single cells from a heterogeneous population.

Once the mechanism of pathway activation is determined, a researcher will be able to develop and test methods to inhibit the pathway. For example, a researcher may measure levels of phosphoryalted Akt, phosphorylated SGK3 to determine whether the PI3K is aberrantly activated in disease cells, and if so, which branch of the pathway is activated. The researcher may also measure levels of nonphosphorylated and/or phosphorylated SGK1 to determine whether pathways parallel to the PI3K-Akt pathway may be activated. The researcher may also measure levels of phosphorylated RTKs such as EGFR, HER2, and KIT to determine whether the aberrant activation occurs upstream or downstream of the receptor. The researcher may then select one or more candidate therapeutics or combinations of therapeutics to inhibit the aberrant PI3K and/or mTOR signaling, treat cell samples with these therapeutics, and measure levels of phosphorylated proteins. By correlating different treatment regimens with inhibition of signaling pathways, the researcher may identify the treatments with the greatest therapeutic efficacy.

One embodiment of the present invention discloses ways of using flow cytometry technologies, including, but not limited to multiparametric flow cytometry and multiparametric phosphoflow cytometry to assist in the development of specific inhibitors of PI3K and/or mTOR signaling components, for example specific inhibitors of p110 isoforms. These inhibitors may be either single agents or in combination with other targeted therapies. One method that will be useful is multiparametric phosphoflow technology which can measure the activity of multiple pathways simultaneously. Other methods which allow the researcher to detect multiple signaling pathways will also be useful.

In one or more of the following non-limiting embodiments, the present invention can be achieved by performing the active steps below and matching cell treatments with the resultant phenotype. Using this method, single-agent therapies or combinations of therapies can be evaluated for activity, dosing, scheduling, or efficacy. Furthermore, these methods may be used for a diagnosis or prognosis. Assessments of signaling activity in patient cells during the course of treatment may be used to monitor patient progress, or to identify the development of drug resistance. The active steps can include:
a. inducing PI3K and/or mTOR signaling by treating cells with a modulator or a combination of modulators, which may include: growth factors, cytokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrates, proteases, free radicals, or complex and undefined biologic compositions which may comprise cellular or botanical extracts, cellular or glandular secretions, or physiologic fluids such as serum, amniotic fluid, or venom;
b. monitoring the activity of gain-of-function mutations in PI3K and/or mTOR, for example in the p110α subunit, by measuring, in single cells, the phosphorylation of downstream substrates. The cells may be untreated or contacted with one or more modulators. The downstream substrates may include, but are not limited to amino acids or post translationally modified amino acids on PDK-1, Akt, MDM2, mTOR, GSK3β, NF-κB, FKHR, BAD, pPRAS40, TSC2, p70S6K, S6, lipin 1, TIF-IA, HIF1α, and 4EBP1, and also phospholipids, including but not limited to PIP2 and PIP3. In addition, components of the apoptotic and proliferative cellular machinery may be monitored;
c. monitoring expression and/or activity levels, at the single cell level, of receptor tyrosine kinases that may activate the PI3K and/or mTOR pathway through increased expression levels and/or gain-of-function mutations. The monitoring may include measuring single-cell expression levels of the receptor tyrosine kinases, phosphorylated forms of the receptor tyrosine kinases, or activated isoforms of elements in pathways parallel to and downstream of these kinases including, but not limited to, the PI3K-Akt pathway, and the Ras-Raf-Erk pathway. The monitoring may be conducted in untreated cells and/or in cells with an evoked signaling response, such as with a modulator. The modulator can be an inhibitor to PI3K, an inhibitor to mTOR, or an inhibitor to both PI3K and mTOR, or an inhibitor to any other protein along the PI3K and/or mTOR pathways. More than one modulator can be used at a given time;
d. monitoring expression and/or phosphorylation or other types of post translational modification levels, at the single cell level, of PI3K and/or mTOR regulatory molecules, including but not limited to, phospholipids, p85 adaptor proteins, GAB, BCAP, IRS-1, IRS-2, (see, e.g. Taniguchi, C. M., et al. Complementary roles of IRS-1 and IRS-2 in the hepatic regulation of metabolism. J. Clin Invest. (2005) 115: 718-27), p70S6K, PTEN, regulators of PTEN (for example, PICT-1, NEDD-4, and DJ-1; see Maehama, T. PTEN: Its Deregulation and Tumorigenesis. Biol. Pharm. Bull. (2007) 30: 1624-27); it is possible that levels of expression alone, levels of post-translational modification, including but not limited to phosphyrylation alone, or levels of both post translational modification and expression of these molecules may be indicative of a disease state or predictive of a clinical outcome. The monitoring may be conducted in untreated cells and/or in cells with an evoked signaling response, such as with a modulator. The modulator can be an inhibitor to PI3K, an inhibitor to mTOR, or an inhibitor to both PI3K and mTOR, or an inhibitor to any other protein along the PI3K and/or mTOR pathways. More than one modulator can be used at a given time. Alternatively, a modulator need not be given;
e. monitoring expression and/or phosphorylation or other types of post translational modification levels, at the single cell level, of mTOR, mTOR substrates, and/or mTOR regulatory molecules, including but not limited to, TSC1, raptor, PRAS40, deptor, gβL, p-S6K, p-p70S6K, lipin-1, HIF1α, eIF4E binding proteins, TIF-IA, rictor, protor, mSIN1, Akt, PDK1, PI3K, SGK1, any PKC isoform, PTEN, regulators of PTEN (for example, PICT-1, NEDD-4, and DJ-1), paxillin, and IRS-1. Levels of expression alone, levels of post translational modification, including but not limited to phosphorylation alone, or levels of both activity and expression of these molecules may be indicative of a disease state or predictive of a clinical outcomeThe monitoring may be conducted in untreated cells and/or in cells with an evoked signaling response, such as with a modulator. The modulator can be an inhibitor to PI3K, an inhibitor to mTOR, or an inhibitor to both PI3K and mTOR, or an inhibitor to any other protein along the PI3K and/or mTOR pathways. More than one modulator can be used at a given time. Alternatively, a modulator need not be given;
f. The monitoring may be conducted in untreated cells and/or in cells treated with a modulator. The monitoring may also be conducted by treating cells with any modulator of mTOR activity, including but not limited to, rapamycin, temsirolimus, everolimus, and/or any other rapamycin analog, PP242, Torinl, WYE-354, Ku-0063794, and/or any other mTOR kinase activity inhibitor. Alternatively, a modulator need not be given;
g. correlating the activity and levels of PI3K and/or mTOR regulatory molecules with clinical outcomes and therapeutic efficacy (ie, compensatory effects might be found in relationships between and among the various components) to identify expression and activity profiles that predict clinical outcome and response to therapies
h. correlating the activity and levels of mTOR and/or mTOR regulatory molecules with clinical outcomes and therapeutic efficacy (ie, compensatory effects might be found in relationships between and among the various components) to identify expression and activity profiles that predict clinical outcome and response to therapies;
i. monitoring epigenetic changes, including but not limited to, methylation, acetylation, that regulate levels and activity of PI3K and/or mTOR regulatory proteins, including but not limited to p85 adapter proteins, GAB, BCAP, IRS-1, IRS-2, PTEN, PICT-1, NEDD-4, DJ-1, and p70S6K by measuring the phosphorylation of PI3K and/or mTOR substrates as described above in a) and b);
j. monitoring expression patterns and activity patterns of microRNAs that regulate the levels and activity of PI3K and/or mTOR pathway elements or PI3K regulatory proteins, including but not limited to, p85, GAB, BCAP, PTEN and p70S6K;
k. measuring the changes in e. and f. and correlating those results with measurements of post translational modifications of PI3K and/or mTOR signaling and regulatory proteins, and elements of pathways parallel to and downstream of PI3K and/or mTOR, including but not limited to, phosphorylation, acetylation, methylation, ubiquitination, sumoylation, that regulate their expression and activity at the single cell level. These measurements can be done in cells, for example somatic or germ line cells that have or are suspected to have a mutational change, such as epigenetic mutations;
l. contacting cells with modulators and measuring changes in expression and activity of p110 isoforms at the single cell level; p110 activity may be inferred by measuring the phosphorylation of substrates downstream of PI3K and/or mTOR, as described in a) and b)
m. measuring changes in expression and activity of PI3K and/or mTOR regulatory molecules at the single cell level, including but not limited to p85, GAB, IRS-1, IRS-2, BCAP, PTEN and p70S6K in the absence and presence of an extracellular modulator, where p110 isoform pathway activity is measured as described by measuring phosphorylation of inositol lipids in a) and b); and
n. performing the above measurements using one or more of the following techniques: flow cytometry, cell imaging, mass spectrometry-based flow cytometry, reverse-transcriptase PCR, microarray analysis, thin layer chromatography, or other methods for measuring phospholipids.

One embodiment of the invention involves methods for monitoring response of neoplasias to drugs specifically designed to correct the molecular abnormalities. Some methods can be useful to select dose and scheduling of these drugs in these patients.

These methods can then be employed to create test-specific assays and kits to determine patient response to drugs that target signaling pathway components, for example RTKs, p110 isoforms, PDK-1, p-Akt isoforms, mTOR, p70S6 kinase, the cell cycle, or apoptosis.

In one embodiment, the cell basal levels may be measured for the proteins or protein modifications of interest, a cellular response induced, and the protein levels measured following the evoked response.

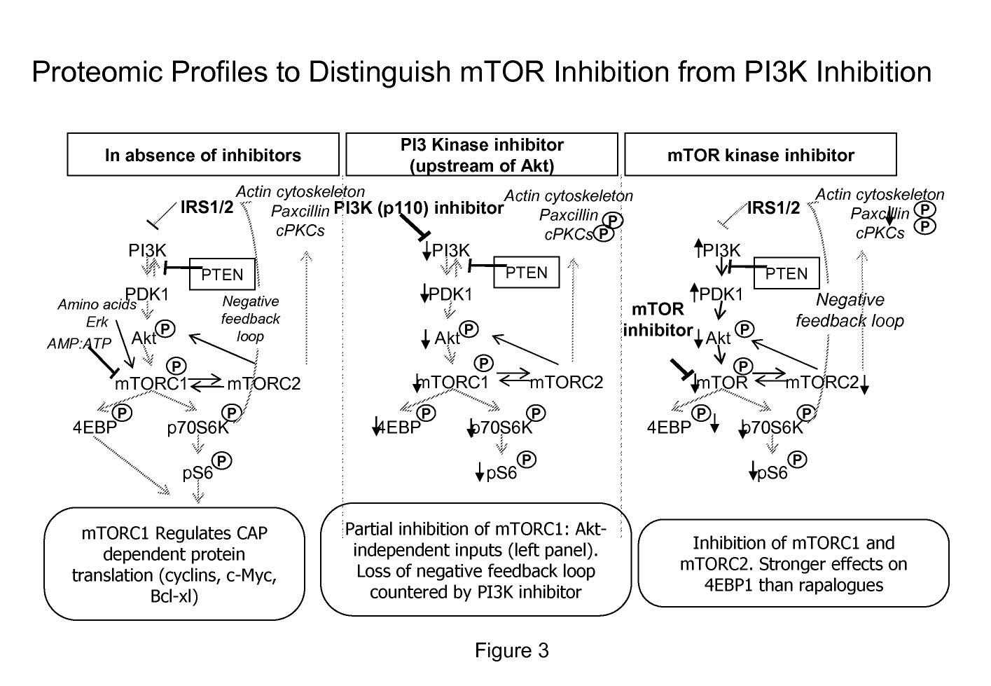

Another embodiment of the present invention can distinguish mTOR inhibition from PI3 kinase inhibition. FIG. 3 shows that mTOR exists in two complexes, TOR complex 1 (TORC1) and TOR complex 2 (TORC2). The substrate specificity profiles of each complex is distinct. Whereas TORC1 has a substrate specificity profile that overlaps with that of PI3K, TORC2 has some overlap, but also a profile that is distinct from TORC1 and PI3K. As such, the phosphorylation state of substrates downstream of TORC2 can be used to monitor inhibition of mTOR versus PI3K. The relative levels of cap-dependent and cap-independent translation may also be used to monitor inhibition of mTOR versus PI3K and/or to monitor inhibition of mTOR clustered within TORC1 and/or TORC2.

See FIG. 3, which illustrates TORC2 substrates including the actin cytoskeleton, paxcillin, and cPKCα and β (see also Bhaksar, P. T., and Hay N., The two TORCs and Akt. Dev. Cell 12: 487-502, 2007). TORC2, but not TORC1 mediates paxillin phosphorylation and actin polarization (Jacinto, E., Mammalian TOR complex 2 controls the actin cytoskeleton and is rapamycin insensitive. Nat. Cell Biol. 6: 1122-28, 2004). TORC2, but not TORC1 also phosphorylates various PKC isoforms (Yang and Guan, Cell research (2007) vol 17, p666-681. Guertin et al, Dev Cell vol 11, p 859, 2006). In one embodiment, cells are treated with an inhibitor, and pAkt, p-PRAS, p-p70S6, p-S6, p-4EBP1 levels are measured to determine whether the inhibitor affects PI3 kinase pathway signaling. The same nodes, plus p-Paxcillin and p-PKCα or β are measured to determine whether the inhibitor affects mTOR signaling.

Yet another embodiment of the invention can distinguish any distinct functional consequences of mTOR inhibition mediated by allosteric inhibitiors, such as rapamycin and its analogs, from mTOR inhibition mediated by mTOR kinase inhibitors, such as PP242, Torinl, WYE-354, Ku-0063794, and the like. Allosteric mTOR inhibitors and mTOR kinase inhibitors target distinct regions of the mTOR protein and modulate cellular signaling in different ways. Rapamycin and its analogs inhibit mTOR within TORC1 while mTOR kinase inhibitors inhibit mTOR within both TORC1 and TORC2. TORC1 and TORC2 have distinct, yet partially overlapping substrate specificity profiles as discussed above, and this difference in substrate specificity combined with the distinct intramolecular targets of mTOR allosteric inhibitors and mTOR kinase inhibitors may allow the determination of distinct functions for the TORC1 and TORC2 multiprotein complexes.

### Samples and Sampling

The methods involve analysis of one or more samples from an individual. An individual is any multicellular organism; in some embodiments, the individual is an animal, e.g., a mammal. In some embodiments, the individual is a human.

The sample may be any suitable type that allows for the analysis of single cells. Samples may be obtained once or multiple times from an individual. Multiple samples may be obtained from different locations in the individual (e.g., blood samples, bone marrow samples and/or lymph node samples), at different times from the individual (e.g., a series of samples taken to monitor response to treatment or to monitor for return of a pathological condition), or any combination thereof. These and other possible sampling combinations based on the sample type, location, and time of sampling allows for the detection of the presence of pre-pathological or pathological cells, the measurement of treatment response, and also the monitoring for disease.

When samples are obtained as a series, e.g., a series of blood samples obtained after treatment, the samples may be obtained at fixed intervals, at intervals determined by the status of the most recent sample or samples or by other characteristics of the individual, or some combination thereof. For example, samples may be obtained at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for sampling and the availability of sampling facilities, thus approximate intervals corresponding to an intended interval scheme are encompassed by the invention. As an example, an individual who has undergone treatment for a cancer may be sampled (e.g., by blood draw) relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, or during the sampling, sampling intervals may be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, bone marrow aspirate, synovial fluid, cerebrospinal fluid, saliva, sweat, tears, semen, sputum, mucus, menstrual blood, breast milk, urine, lymphatic fluid, amniotic fluid, placental fluid and effusions such as cardiac effusion, joint effusion, pleural effusion, and peritoneal cavity effusion (ascites). Rinses can be obtained from numerous organs, body cavities, passage ways, ducts and glands. Sites that can be rinsed include lungs (bronchial lavage), stomach (gastric lavage), gastrointestinal track (gastrointestinal lavage), colon (colonic lavage), vagina, bladder (bladder irrigation), breast duct (ductal lavage), oral, nasal, sinus cavities, and peritoneal cavity (peritoneal cavity perfusion). In some embodiments the sample or samples is blood.

Solid tissue samples may also be used, either alone or in conjunction with fluid samples. Solid samples may be derived from individuals by any method known in the art including surgical specimens, biopsies, and tissue scrapings, including cheek scrapings. Surgical specimens include samples obtained during exploratory, cosmetic, reconstructive, or therapeutic surgery. Biopsy specimens can be obtained through numerous methods including bite, brush, cone, core, cytological, aspiration, endoscopic, excisional, exploratory, fine needle aspiration, incisional, percutaneous, punch, stereotactic, and surface biopsy.

In some embodiments, the sample is a blood sample. In some embodiments, the sample is a bone marrow sample. In some embodiments, the sample is a lymph node sample. In some embodiments, the sample is cerebrospinal fluid. In some embodiments, combinations of one or more of a blood, bone marrow, cerebrospinal fluid, and lymph node sample are used.

One or more cells or cell types, or samples containing one or more cells or cell types, can be isolated from body samples. The cells can be separated from body samples by centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells may be obtained. Alternatively, a heterogeneous cell population can be used. Cells can also be separated by using filters. For example, whole blood can also be applied to filters that are engineered to contain pore sizes that select for the desired cell type or class. Rare pathogenic cells can be filtered out of diluted, whole blood following the lysis of red blood cells by using filters with pore sizes between 5 to 10 nm, as disclosed in U.S. patent application Ser. No. 09/790,673. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Ser. Nos. 61/048,886; 61/048,920; and 61/048,657. See also, the commercial products from companies such as BD and BCI as identified above.

See also U.S. Pat. Nos. 7,381,535 and 7,393,656. All of the above patents and applications are incorporated by reference as stated above.

In some embodiments, the cells are cultured post collection in a media suitable for revealing the activation level of an activatable element (e.g. RPMI, DMEM) in the presence, or absence, of serum such as fetal bovine serum, bovine serum, human serum, porcine serum, horse serum, or goat serum. When serum is present in the media it could be present at a level ranging from 0.0001% to 30%.

### Compounds to be Analyzed

Compounds that are analyzed in some embodiments of the present invention are designed to treat cancer, immune disorders, metabolic disorders and other diseases. The compounds can also be any of the modulators provided below. In some embodiments, the compounds can induce cell death or apotosis or simply stabilize the disease.

Active compounds include agents that induce cell death or apoptosis. These agents may be common cytotoxic agents that are used in cancer chemotherapy, or any other agents that are just generally toxic to cells. Example agents include targeted therapies, such as small molecules directed to biological targets.

In some embodiments, compounds are small-molecule inhibitors of the PI3 kinase pathway. Many small-molecule inhibitors of the PI3 kinase pathway are actively being developed by various pharmaceutical companies. Examples include LY294002 and its derivatives (see Vlahos, C. (1994) J. Biol. Chem. 269, 5241-5248.)

### Activatable Elements

The methods and compositions of the invention may be employed to examine and profile the status of any activatable element in a cellular pathway, or collections of such activatable elements. Single or multiple distinct pathways may be profiled (sequentially or simultaneously), or subsets of activatable elements within a single pathway or across multiple pathways may be examined (again, sequentially or simultaneously). The cell can be any cell from an individual, including for example, a hematopoietic cell or one which originates from a solid tumor. Examples of hematopoietic cells include, but are not limited to pluripotent hematopoietic stem cells, granulocyte lineage progenitor or derived cells, monocyte lineage progenitor or derived cells, macrophage lineage progenitor or derived cells, megakaryocyte lineage progenitor or derived cells and erythroid lineage progenitor or derived cells. As a non limiting example, the cells may also come from solid tumors as circulating tumor cells, ascites from ovarian cancer, and cells derived from larger masses, such as from biopses. Circulating tumor cells may be rare cells, see U.S. Ser. No. 61/048,886.

In some embodiments, the invention is directed to methods for determining the activation level of one or more activatable elements in a cell before and/or after treatment with one or more modulators. The activation of an activatable element in the cell upon treatment with one or more modulators can reveal operative pathways in a condition that can then be used, e.g., as an indicator to predict the course of the condition, to identify a risk group, to predict an increased risk of developing secondary complications or suffering harmful side effects, to choose a therapy for an individual, to predict response to a therapy for an individual, to determine the efficacy of a therapy in an individual, and to determine the prognosis for an individual.

In some embodiments, the activation level of an activatable element in a cell is determined by contacting the cell with at least 1, 2, 3, 4, 5, 6, 7, 8, 9, or 10 modulators. In some embodiments, the activation level of an activatable element in a cell is determined by contacting the cell with at least 1, 2, 3, 4, 5, 6, 7, 8, 9, or 10 modulators where at least one of the modulators is an inhibitor. In other embodiments, the activation level of an activatable element in a cell is determined by contacting the cell with at least 1, 2, 3, 4, 5, 6, 7, 8, 9, or 10 modulators where at least one of the modulators is an activator. In some embodiments, the activation level of an activatable element in a cell is determined by contacting the cell with an inhibitor and another modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

In some embodiments, a phenotypic profile of a population of cells is determined by measuring the activation level of an activatable element when the population of cells is exposed to a plurality of modulators in separate cultures.

As will be appreciated by those in the art, a wide variety of activation events can find use in the present invention. In general, the basic requirement is that the activation results in a change in the activatable element that is detectable by some indication (termed an “activation state indicator”), preferably by altered binding of a labeled binding element or by changes in detectable biological activities (e.g., the activated state has an enzymatic activity which can be measured and compared to a lack of activity in the non-activated state). What is important is to differentiate, using detectable events or moieties, between two or more activation states.

As an illustrative example, and without intending to be limited to any theory, an individual phosphorylatable site on a protein can activate or deactivate the protein. Additionally, phosphorylation of an adapter protein may promote its interaction with other components/proteins of distinct cellular signaling pathways. The terms “on” and “off,” when applied to an activatable element that is a part of a cellular constituent, are used here to describe the state of the activatable element, and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis for classifying cells is that the distribution of activation levels for one or more specific activatable elements will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to classify cells in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may or may not contain activatable elements, of cell or a population of cells may be used to classify a cell or a population of cells into a class. Once the activation level of intracellular activatable elements of individual single cells is known they can be placed into one or more classes, e.g., a class that corresponds to a phenotype. A class encompasses a class of cells wherein every cell has the same or substantially the same known activation level, or range of activation levels, of one or more intracellular activatable elements. For example, if the activation levels of five intracellular activatable elements are analyzed, predefined classes of cells that encompass one or more of the intracellular activatable elements can be constructed based on the activation level, or ranges of the activation levels, of each of these five elements. It is understood that activation levels can exist as a distribution and that an activation level of a particular element used to classify a cell may be a particular point on the distribution but more typically may be a portion of the distribution.

In addition to activation levels of intracellular activatable elements, levels of intracellular or extracellular biomolecules, e.g., proteins, may be used alone or in combination with activation states of activatable elements to classify cells. Further, additional cellular elements, e.g., biomolecules or molecular complexes such as RNA, DNA, carbohydrates, metabolites, and the like, may be used in conjunction with activatable states or expression levels in the classification of cells encompassed here.

In some embodiments, other characteristics that affect the status of a cellular constituent may also be used to classify a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

Additional elements may also be used to classify a cell, such as the presence or absence of extracellular markers, surface markers, intracellular markers, nuclear antigens, enzymatic activity, protein expression and localization, cell cycle analysis, chromosomal analysis, cell volume, and morphological characteristics like granularity and size of nucleus or other distinguishing characteristics. Non-limiting examples of cell surface markers and intracellular markers include proteins, carbohydrates, lipids, nucleic acids, and metabolites. For example, B cells can be further subdivided based on the expression of cell surface markers such as CD19, CD20, CD22 or CD23. Other non-limiting examples of markers useful for the classification of cells include CD3, CD4, CD8, CD19, CD25, CD33, CD45RA, CD69, and Foxp3. Cells can be categorized for the presence, absence, high level, or low level of one ore more markers. Markers can be used alone or in combination. For example, cells can be classified by using 1, 2, 3, 4, 5, 6, 7, 8, 9, 10, or more markers.

Alternatively, predefined classes of cells can be aggregated or grouped based upon shared characteristics that may include inclusion in one or more additional predefined classes or the presence of extracellular or intracellular markers, similar gene expression profile, nuclear antigens, enzymatic activity, protein expression and localization, cell cycle analysis, chromosomal analysis, cell volume, and morphological characteristics like granularity and size of nucleus or other distinguishing cellular characteristics.

In some embodiments, the physiological status of one or more cells is determined by examining and profiling the activation level of one or more activatable elements in a cellular pathway. In some embodiments, a cell is classified according to the activation level of a plurality of activatable elements. In some embodiments, a cell is classified according to the activation levels of a plurality of activatable elements. In some embodiments, 1, 2, 3, 4, 5, 6, 7, 8, 9, 10 or more activatable elements may be analyzed in a cell signaling pathway. In some embodiments, the activation levels of one or more activatable elements of a cell are correlated with a condition. In some embodiments, the activation levels of one or more activatable elements of a cell are correlated with a neoplastic condition as described herein.

In some embodiments, the activation level of one or more activatable elements in single cells in the sample is determined. Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, degradation through ubiquitination and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation level of one or more activatable elements in single cells in the sample is determined. Activation states of activatable elements may result from chemical additions or modifications of biomolecules and include many biochemical processes. See U.S. Application No. 61/085,789, which is incorporated by reference. Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, degradation through ubiquitination and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, other characteristics that affect the status of a cellular constituent may also be used to classify a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

Additional elements may also be used to classify a cell or to measure the activation state of activatable elements, such as the expression level of extracellular or intracellular markers, nuclear antigens, enzymatic activity, protein expression and localization, cell cycle analysis, chromosomal analysis, cell volume, and morphological characteristics like granularity and size of nucleus or other distinguishing characteristics. The level of cap-dependent translation of any particular protein or combination thereof may be monitored by expressing a reporter construct within a single cell or population of cells. The reporter construct may comprise a bicistronic reporter vector that expresses a first fluorescent protein from a cap-dependent promoter and a second fluorescent protein from a non-cap-dependent promoter, such as an internal ribosome entry site (IRES). Preferably, the first and the second fluorescent proteins have different emission wavelengths. The relative levels of cap-dependent and cap-independent translation may be determined by comparing the fluorescent intensities of the first and the second fluorescent proteins. The level of cap-dependent translation of any particular protein or combination thereof may also be monitored by using a fluorophore-labled antisense nucleic acid strand that may specifically hybridize to the mRNA transcript of any gene and/or protein of interest. The fluorophore-labled antisense nucleic acid strand may be detected using various methods described below.

In some embodiments, the activation levels of a plurality of intracellular activatable elements in single cells are determined. In some embodiments, at least about 1, 2, 3, 4, 5, 6, 7, 8, 9, or more than 10 intracellular activatable elements are determined.

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

One example of a covalent modification is the substitution of a phosphate group for a hydroxyl group in the side chain of an amino acid (phosphorylation). A wide variety of proteins are known that recognize specific protein substrates and catalyze the phosphorylation of serine, threonine, or tyrosine residues on their protein substrates. Such proteins are generally termed “kinases.” Substrate proteins that are capable of being phosphorylated are often referred to as phosphoproteins (after phosphorylation). Once phosphorylated, a substrate phosphoprotein may have its phosphorylated residue converted back to a hydroxylated residue by the action of a protein phosphatase that specifically recognizes the substrate protein. Protein phosphatases catalyze the replacement of phosphate groups by hydroxyl groups on serine, threonine, or tyrosine residues. Through the action of kinases and phosphatases a protein may be reversibly phosphorylated on a multiplicity of residues and its activity may be regulated thereby. Thus, the presence or absence of one or more phosphate groups in an activatable protein is a preferred readout in the present invention.

Another example of a covalent modification of an activatable protein is the acetylation of histones. Through the activity of various acetylases and deacetylylases the DNA binding function of histone proteins is tightly regulated. Furthermore, histone acetylation and histone deactelyation have been linked with malignant progression. See Nature, 429: 457-63, 2004.

Another form of activation involves cleavage of the activatable element. For example, one form of protein regulation involves proteolytic cleavage of a peptide bond. While random or misdirected proteolytic cleavage may be detrimental to the activity of a protein, many proteins are activated by the action of proteases that recognize and cleave specific peptide bonds. Many proteins derive from precursor proteins, or pro-proteins, which give rise to a mature isoform of the protein following proteolytic cleavage of specific peptide bonds. Many growth factors are synthesized and processed in this manner, with a mature isoform of the protein typically possessing a biological activity not exhibited by the precursor form. Many enzymes are also synthesized and processed in this manner, with a mature isoform of the protein typically being enzymatically active, and the precursor form of the protein being enzymatically inactive. This type of regulation is generally not reversible. Accordingly, to inhibit the activity of a proteolytically activated protein, mechanisms other than “reattachment” must be used. For example, many proteolytically activated proteins are relatively short-lived proteins, and their turnover effectively results in deactivation of the signal Inhibitors may also be used. Among the enzymes that are proteolytically activated are serine and cysteine proteases, including cathepsins and caspases respectively. Many other proteolytically activated enzymes, known in the art as “zymogens,” also find use in the instant invention as activatable elements.

In an alternative embodiment, the activation of the activatable element involves prenylation of the element. By “prenylation”, and grammatical equivalents used herein, is meant the addition of any lipid group to the element. Common examples of prenylation include the addition of farnesyl groups, geranylgeranyl groups, myristoylation, and palmitoylation. In general these groups are attached via thioether linkages to the activatable element, although other attachments may be used.

In one embodiment, the activatable enzyme is a caspase. The caspases are an important class of proteases that mediate programmed cell death (referred to in the art as “apoptosis”). Caspases are constitutively present in most cells, residing in the cytosol as a single chain proenzyme. These are activated to fully functional proteases by a first proteolytic cleavage to divide the chain into large and small caspase subunits and a second cleavage to remove the N-terminal domain. The subunits assemble into a tetramer with two active sites (Green, Cell 94:695-698, 1998). Many other proteolytically activated enzymes, known in the art as “zymogens,” also find use in the instant invention as activatable elements.

In alternative embodiment, activation of the activatable element is detected as intermolecular clustering of the activatable element. By “clustering” or “multimerization”, and grammatical equivalents used herein, is meant any reversible or irreversible association of one or more signal transduction elements. Clusters can be made up of 2, 3, 4, etc., elements. Clusters of two elements are termed dimers. Clusters of 3 or more elements are generally termed oligomers, with individual numbers of clusters having their own designation; for example, a cluster of 3 elements is a trimer, a cluster of 4 elements is a tetramer, etc.

Clusters can be made up of identical elements or different elements. Clusters of identical elements are termed “homo” dimers, while clusters of different elements are termed “hetero” clusters. Accordingly, a cluster can be a homodimer, as is the case for the β₂-adrenergic receptor.

Alternatively, a cluster can be a heterodimer, as is the case for GABA_{B-R}. In other embodiments, the cluster is a homotrimer, as in the case of TNFα, or a heterotrimer such the one formed by membrane-bound and soluble CD95 to modulate apoptosis. In further embodiments the cluster is a homo-oligomer, as in the case of Thyrotropin releasing hormone receptor, or a hetero-oligomer, as in the case of TGFβ1. One embodiment includes hetero and homo dimmers of the EGF receptor (HER) family of receptor tyrosine kinases.

In a preferred embodiment, the activation or signaling potential of elements is mediated by clustering, irrespective of the actual mechanism by which the element's clustering is induced. For example, elements can be activated to cluster a) as membrane bound receptors by binding to ligands (ligands including both naturally occurring or synthetic ligands), b) as membrane bound receptors by binding to other surface molecules, or c) as intracellular (non-membrane bound) receptors binding to ligands.

In a preferred embodiment the activatable elements are membrane bound receptor elements that cluster upon ligand binding such as cell surface receptors. As used herein, “cell surface receptor” refers to molecules that occur on the surface of cells, interact with the extracellular environment, and transmit or transduce (through signals) the information regarding the environment intracellularly in a manner that may modulate cellular activity directly or indirectly, e.g., via intracellular second messenger activities or transcription of specific promoters, resulting in transcription of specific genes. One class of receptor elements includes membrane bound proteins, or complexes of proteins, which are activated to cluster upon ligand binding. As is known in the art, these receptor elements can have a variety of forms, but in general they comprise at least three domains. First, these receptors have a ligand-binding domain, which can be oriented either extracellularly or intracellularly, usually the former. Second, these receptors have a membrane-binding domain (usually a transmembrane domain), which can take the form of a seven pass transmembrane domain (discussed below in connection with G-protein-coupled receptors) or a lipid modification, such as myristylation, to one of the receptor's amino acids which allows for membrane association when the lipid inserts itself into the lipid bilayer. Finally, the receptor has a signaling domain, which is responsible for propagating the downstream effects of the receptor.

Examples of such receptor elements include but are not limited to hormone receptors, steroid receptors, cytokine receptors, such as IL1-α, IL-β, IL-2, IL-3, IL-4, IL-5, IL-6, IL-7, IL-8, IL-9, IL-10. IL-12, IL-15, IL-18, IL-21, CCR5, CCR7, CCR-1-10, CCL20, chemokine receptors, such as CXCR4, adhesion receptors and growth factor receptors, including, but not limited to, PDGF-R (platelet derived growth factor receptor), EGF-R (epidermal growth factor receptor), VEGF-R (vascular endothelial growth factor), fibroblast growth factor receptor, uPAR (urokinase plasminogen activator receptor), ACHR (acetylcholine receptor), IgE-R (immunoglobulin E receptor), estrogen receptor, thyroid hormone receptor, integrin receptors β1, β2, β3, β4, β5, β6, α1, α2, α3, α 4, α 5, α 6), MAC-1 (β2 and cd1 1b), αVβ33, opioid receptors (mu and kappa), FC receptors, serotonin receptors (5-HT, 5-HT6, 5-HT7), β-adrenergic receptors, insulin receptor, leptin receptor, tumor necrosis factor (TNF) receptor, tnf family member receptors, statin receptors, FAS receptor, BAFF receptor, APRIL receptor, FLT3 Ligand receptor, Stem cell factor receptor, GM-CSF receptor, G-CSF receptor, erythropoietin (EPO) receptor, and thrombopoietin receptor.

In one embodiment, the activatable element is a molecule in the PI3 kinase pathway. See FIG. 1 for examples.

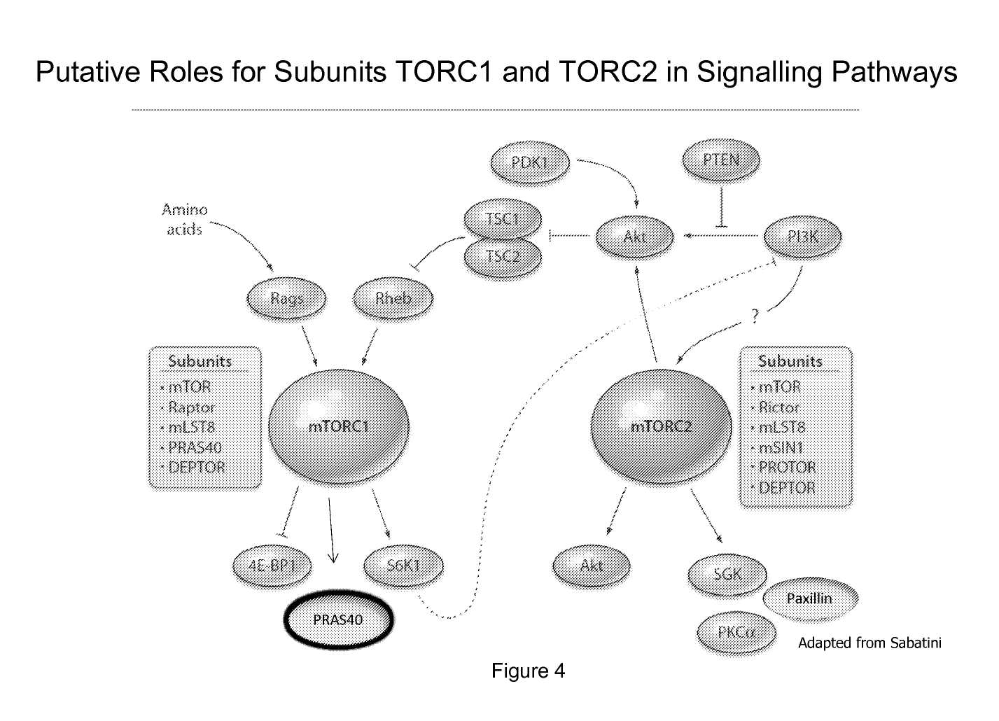

In another embodiment, the activatable element is a molecule in any mTOR kinase pathway. See FIG. 4 for examples.

The receptor tyrosine kinases can be divided into subgroups on the basis of structural similarities in their extracellular domains and the organization of the tyrosine kinase catalytic region in their cytoplasmic domains. Sub-groups I (epidermal growth factor (EGF) receptor-like), II (insulin receptor-like) and the EPH/ECK family contain cysteine-rich sequences (Hirai et al., (1987) Science 238:1717-1720 and Lindberg and Hunter, (1990) Mol. Cell. Biol. 10:6316-6324). The functional domains of the kinase region of these three classes of receptor tyrosine kinases are encoded as a contiguous sequence (Hanks et al. (1988) Science 241:42-52). Subgroups III (platelet-derived growth factor (PDGF) receptor-like) and IV (the fibroblast growth factor (FGF) receptors) are characterized as having immunoglobulin (Ig)-like folds in their extracellular domains, as well as having their kinase domains divided in two parts by a variable stretch of unrelated amino acids (Yarden and Ullrich (1988) supra and Hanks et al. (1988) supra). For further discussion, see U.S. Patent Application 61/120,320.

In a further embodiment, the receptor element is an integrin other than Leukocyte Function Antigen-1 (LFA-1). Members of the integrin family of receptors function as heterodimers, composed of various α and β subunits, and mediate interactions between a cell's cytoskeleton and the extracellular matrix. (Reviewed in, Giancotti and Ruoslahti, Science 285, 13 Aug. 1999). Different combinations of the α and β subunits give rise to a wide range of ligand specificities, which may be increased further by the presence of cell-type-specific factors. Integrin clustering is known to activate a number of intracellular pathways, such as the RAS, Rab, MAP kinase pathway, and the PI3 kinase pathway. In a preferred embodiment the receptor element is a heterodimer (other than LFA-1) composed of a 0 integrin and an α integrin chosen from the following integrins; β1, β2, β3, β4, β5, β6, α1, α2, α3, α4, α5, and α6, or is MAC-1 (β2 and cd11b), or αVβ3.

In a preferred embodiment the element is an intracellular adhesion molecule (ICAM). ICAMs-1, -2, and -3 are cellular adhesion molecules belonging to the immunogloblin superfamily. Each of these receptors has a single membrane-spanning domain and all bind to β2 integrins via extracellular binding domains similar in structure to Ig-loops. (Signal Transduction, Gomperts, et al., eds, Academic Press Publishers, 2002, Chapter 14, pp 318-319).

In another embodiment the activatable elements cluster for signaling by contact with other surface molecules. In contrast to the receptors discussed above, these elements cluster for signaling by contact with other surface molecules, and generally use molecules presented on the surface of a second cell as ligands. Receptors of this class are important in cell-cell interactions, such mediating cell-to-cell adhesion and immunorecognition.

Examples of such receptor elements are CD3 (T cell receptor complex), BCR (B cell receptor complex), CD4, CD28, CD80, CD86, CD54, CD102, CD50 and ICAMs 1, 2 and 3.

In one embodiment, the activatable elements are intracellular receptors capable of clustering. Elements of this class are not membrane-bound. Instead, they are free to diffuse through the intracellular matrix where they bind soluble ligands prior to clustering and signal transduction. In contrast to the previously described elements, many members of this class are capable of binding DNA after clustering to directly effect changes in RNA transcription.

In another embodiment the intracellular receptors capable of clustering are perioxisome proliferator-activated receptors (PPAR). PPARs are soluble receptors responsive to lipophillic compounds, and induce various genes involved in fatty acid metabolism. The three PPAR subtypes, PPAR α, β, and γ have been shown to bind to DNA after ligand binding and heterodimerization with retinoid X receptor. (Summanasekera, et al., J Biol Chem, M211261200, Dec. 13, 2002.)

In another embodiment the activatable element is a nucleic acid. Activation and deactivation of nucleic acids can occur in numerous ways including, but not limited to, cleavage of an inactivating leader sequence as well as covalent or non-covalent modifications that induce structural or functional changes. For example, many catalytic RNAs, e.g. hammerhead ribozymes, can be designed to have an inactivating leader sequence that deactivates the catalitic activity of the ribozyme until cleavage occurs. An example of a covalent modification is methylation of DNA. Deactivation by methylation has been shown to be a factor in the silencing of certain genes, e.g. STAT regulating SOCS genes in lymphomas. See Leukemia. See February 2004; 18(2): 356-8. SOCS1 and SHPT hypermethylation in mantle cell lymphoma and follicular lymphoma: implications for epigenetic activation of the Jak/STAT pathway. Chim C S, Wong K Y, Loong F, Srivastava G.

In another embodiment, the activatable element is a microRNA. MicroRNAs (miRNAs) are non-coding RNA molecules, approximately 22 nucleotides in length, which play important regulatory roles in gene expression in animals and plants. MiRNAs modulate gene flow through post-transcriptional gene silencing through the RNA interference pathway. The net effect is to downregulate the expression of the target gene by preventing the protein product from being produced. Mirnezami et al., MicroRNAs: Key players in carcinogenesis and novel therapeutic agents, Eur. J. Surg. Oncol., Jun. 9, 2006, doi:10.1016/j.ejso.2008.06.006, hereby fully incorporated by reference in its entirety.

The discovery of a novel class of gene regulators, named microRNAs (miRNAs), has changed the landscape of human genetics miRNAs are ˜22 nucleotide non-coding RNA that regulate gene expression by binding to 3′ untranslated regions of mRNA. Once one strand of miRNA is incorporated into the RNA induced silencing complex (RISC), it interacts with the 3′ untranslated regions (UTRs) of target mRNAs through partial sequence complementarity to bring about translational repression or mRNA degradation. If there is perfect complementarity, the mRNA is cleaved and degraded whereas if the base pairing is imperfect, translational silencing is the main mechanism. Recent work has led to an increased understanding of the role of miRNAs in hematopoietic differentiation and leukemogenesis. Using animal models engineered to overexpress miR-150, miR-17 approximately 92 and miR-155 or to be deficient for miR-223, miR-155 and miR-17 approximately 92 expression, several groups have now shown that miRNAs are critical for B-lymphocyte development (miR-150 and miR-17 approximately 92), granulopoiesis (miR-223), immune function (miR-155) and B-lymphoproliferative disorders (miR-155 and miR-17 approximately 92). Distinctive miRNA signatures have been described in association with cytogenetics and outcome in acute myeloid leukemia. There is now strong evidence that miRNAs modulate not only hematopoietic differentiation and proliferation but also activity of hematopoietic cells, in particular those related to immune function. Extensive miRNA deregulation has been observed in leukemias and lymphomas and mechanistic studies support a role for miRNAs in the pathogenesis of these disorders (Garzon et al, MicroRNAs in normal and malignant hematopoiesis, Current Opinion Hematology, 2008, 15:352-8). miRNAs regulate critical cellular processes such as cell cycle, apoptosis and differentiation. Consequently impairments in their regulation of these functions through changes in miRNA expression can lead to tumorigenesis miRNAs can act as oncogenes or tumor suppressors miRNA profiles can provide important prognostic information as recently shown for acyute myeloid leukemia (Marcucci et al., J. Clinical Oncology (2008) 26:p5078). In another study, Cimmino et al., (PNAS (2005) 102:p. 13944) showed that patients with chronic lymphocytic leukemia (CLL) have deletions or down regulation of two clustered miRNA genes; mir-15a and mir-16-1. These miRNAs negatively regulate the anti-apoptotic protein Bcl-2 that is often overexpressed in multiple cancers including but not limited to leukemias and lymphomas. Thus, miRNAs are a potentially useful diagnostic tool in diagnosing cancer, classifying different types of tumors, and determining clinical outcome, including but not limited to, MPNs. A. Esquela-Kerscher and F. J. Slack, Oncomirs—microRNAs with a role in cancer, Nat. Rev. Cancer, Apr. 2006, 6: 259-269 is hereby fully incorporated by reference.

In another embodiment the activatable element is a small molecule, carbohydrate, lipid or other naturally occurring or synthetic compound capable of having an activated isoform. In addition, as pointed out above, activation of these elements need not include switching from one form to another, but can be detected as the presence or absence of the compound. For example, activation of cAMP (cyclic adenosine mono-phosphate) can be detected as the presence of cAMP rather than the conversion from non-cyclic AMP to cyclic AMP.

Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference. See also U.S. Ser. Nos. 61/048,886; 61/048,920; and Shulzet al., Current Protocols in Immunology 2007, 78:8.17.1-20.

In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIEL TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6 Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3a, GSK3I3, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon (3, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bc1-XL, Bcl-w, Bcl-B, Al, Bax, Bak, Bok, Bik, Bad, Bid, Bim, Bmf, Hrk, Noxa, Puma, IAPB, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pinl prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFκB), CREB, NFAT, ATF-2, AFT, Myc, Fos, Spl, Egr-1, T-bet, β-catenin, HIFs, FOXOs, E2Fs, SRFs, TCFs, Egr-1, katenin, FOXO STAT1, STAT 3, STAT 4, STAT 5, STAT 6, p53, WT-1, HMGA, pS6, 4EPB-1, eIF4E-binding protein, RNA polymerase, initiation factors, elongation factors.

Generally, the methods of the invention involve determining the activation levels of an activatable element in a plurality of single cells in a sample. The activation levels can be obtained by perturbing the cell state using a modulator.

### Modulators

In some embodiments, the methods and composition utilize a modulator. A modulator can be an activator, a therapeutic compound, an inhibitor or a compound capable of impacting a cellular pathway or causing an effect in an activatable element, or some combination of the above. Modulators can also take the form of a variety of environmental cues and inputs.

In some embodiments, the methods and compositions utilize a modulator. A modulator can be an activator, a therapeutic agent, an inhibitor or a compound capable of impacting cellular signaling networks. Modulators can take the form of a wide variety of environmental cues and inputs. Modulators can be specific for cell types, such as a cancer cell modulator or a hematopoietic cell modulator. Examples of modulators include, but are not limited to a: growth factor, chemokine, cytokine, drug, immune modulator, ion, neurotransmitter, adhesion molecule, hormone, small molecule, inorganic compound, polynucleotide, antibody, natural compound, lectin, lactone, chemotherapeutic agent, biological response modifier, carbohydrate, protease, free radical, complex and undefined biologic composition, cellular secretion, glandular secretion, physiologic fluid, reactive oxygen species, virus, electromagnetic radiation, ultraviolet radiation, infrared radiation, particulate radiation, redox potential, pH modifier, the presence or absences of a nutrient, change in temperature, change in oxygen partial pressure, change in ion concentration or application of oxidative stress, physical parameter such as heat, cold, UV radiation, peptide, and protein fragment, either alone or in the context of cells, cells themselves, viruses, and biological and non-biological complexes (e.g. beads, plates, viral envelopes, antigen presentation molecules such as major histocompatibility complex). Examples of modulators include but are not limited to IL-1, IL-2, IL-3, IL-4, IL-6, IL-7, IL-10, IL-12, IL-15, IL-21, IL-27, GM-CSF, G-CSF, IFNα, IFNγ, T cell recepter (TCR) cross-linking antibodies, B cell receptor (BCR) cross-linking antibodies SDF-1α, FLT-3L, IGF-1, M-CSF, SCF, PMA, Thapsigargin, H202, etoposide, AraC, daunorubicin, staurosporine, benzyloxycarbonyl-Val-Ala-Asp (OMe) fluoromethylketone (ZVAD), lenalidomide, EPO, azacitadine, decitabine, LPS, TNF-α, and CD4OL. In some embodiments, the modulator is an activator. In some embodiments the modulator is an inhibitor. In some embodiments, the modulators include growth factors, cytokines, chemokines, phosphatase inhibitors, and pharmacological reagents. The response panel is composed of at least one of: IL-1, IL-2, IL-3, IL-4, IL-6, IL-7, IL-10, IL-12, IL-15, IL-21, IL-27, GM-CSF, G-CSF, IFNα, IFNy, T cell recepter cross-linking antibodies, B cell receptor cross-linking antibodies SDF-1a, FLT-3L, IGF-1, M-CSF, SCF, PMA, Thapsigargin, H202, etoposide, AraC, daunorubicin, staurosporine, benzyloxycarbonyl-Val-Ala-Asp (OMe) fluoromethylketone (ZVAD), lenalidomide, EPO, azacitadine, decitabine, LPS, TNF-a, and CD4OL. Examples of TCR crosslinking antibodies include, but are not limited to, anti-CD3 and anti CD28 antibodies. Examples of BCR crosslinking antibodies include, but are not limited to, anti-IgG, anti-IgM, anti-kappa, and anti-lambda antibodies.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators. See the U.S. Patent Applications recited above which are incorporated by reference, such as U.S. Ser. No. 61/120,320.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrates, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments, the modulator is an inhibitor. In some embodiments, the inhibitor is an inhibitor of a cellular factor or a plurality of factors that participates in a cellular pathway (e.g. signaling cascade) in the cell. In some embodiments, the inhibitor is a phosphatase inhibitor. Examples of phosphatase inhibitors include, but are not limited to H₂0₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenylarsine oxide, Pyrrolidine Dithiocarbamate, and Aluminium fluoride.

Modulators can be specific PI3K inhibitors. Specific examples of such PI3K inhibitors include SF-1126 (Semafore Pharmaceuticals), BEZ-235 (Novartis), XL-147 (Exelixis, Inc.), and GDC-0941 (Genentech, Inc.). Modulators can also be general PI3K inhibitors. Other known PI3K inhibitors include celecoxib and analogs thereof, such as OSU-03012 and OSU-03013 (e.g., Zhu et al., Cancer Res., 64(12): 4309-18, 2004); 3-deoxy-D-myo-inositol analogs (e.g., U.S. Application No. 20040192770; Meuillet et al., Oncol. Res., 14:513-27, 2004); fused heteroaryl derivatives (U.S. Pat. No. 6,608,056); 3-(imidazo[1,2-a]pyridin-3-yl) derivatives (e.g., U.S. Pat. Nos. 6,403,588 and 6,653,320); Ly294002 (e.g., Vlahos, et al., J. Biol., Chem., 269(7) 5241-5248, 1994); quinazoline-4-one derivatives, such as IC486068 (e.g., U.S. Application No. 20020161014, Geng et al., Cancer Res., 64:4893-99, 2004); 3-(hetero)aryloxy substituted benzo(b)thiophene derivatives (e.g., WO 04 108715; also WO 04 108713); viridins, including semi-synthetic viridins (e.g., Ihle et al., Mol Cancer Ther., 3(7):763-72, 2004; U.S. Application No. 20020037276; U.S. Pat. No. 5,726,167); and wortmannin and derivatives thereof (e.g., U.S. Pat. Nos. 5,504,103; 5,480,906, 5,468,773; 5,441,947; 5,378,725; and 3,668,222). Modulators can also be specific mTOR inhibitors. Examples of mTOR inhibitors include everolimus (RAD001, Novartis), zotarolimus (Abbott), temsirolimus (CCI-779, Wyeth), AP 23573 (Ariad), AP23675, Ap23841, TAFA 93, rapamycin (sirolimus) and combinations thereof. Modulators can also be inhibitors that inhibit both PI3K and mTOR. Alternatively, modulators can inhibit any PI3K and/or mTOR pathway protein, either specifically or by inhibiting more than one PI3K and/or mTOR pathway protein. For example, pyrimidyl cyclopentanes can be used as Akt protein kinase inhibitors (see US Patent Publication No. 20080058327, which is hereby incorporated by reference in its entirety). Other known inhibitors are disclosed in US Patent Publication Nos. 20070259876, 20080051399, 20090247567 and 20090318411. Each of the above mentioned references are hereby incorporated in their entirety.

In some embodiments, the modulator is an anti-tumor or anti-cancer agent. Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin; alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present invention can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126, and Zosuquidar; Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; Taxol; thiosemicarbazone derivatives; telomerase inhibitors; arsenic trioxide; planomycin; sulindac sulfide; cyclopamine; purmorphamine; gamma-secretase inhibitors; CXCR4 inhibitors; HH signaling inhibitors; Bmi-1 inhibitors; Bc1-2 inhibitors; Notch-1 inhibitors; DNA checkpoint protein inhibitors; ABC transporter inhibitors; mitotic inhibitors; intercalating antibiotics; growth factor inhibitors; cell cycle modulators; enzymes; topoisomerase inhibitors; biological response modifiers; angiogenesis inhibitors; DNA repair inhibitors; and small G-protein inhibitors. Combinations can be made with one or more than one of the above.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the cross-linker is a molecular binding entity. In some embodiments, the molecular binding entity is monovalent, bivalent, or multivalent and may be made more multivalent by attachment to a solid surface or tethered on a nanoparticle surface to increase the local valency of the epitope binding domain.

In some embodiments, the inhibitor is an inhibitor of a cellular factor or a plurality of factors that participates in a cellular pathway (e.g. signaling cascade) in the cell. In some embodiments, the inhibitor is a phosphatase inhibitor.

In some embodiments, the activation level of an activatable element in a cell is determined by contacting the cell with an inhibitor and a modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators

In one embodiment, the modulators are FLT3L, G-CSF, GM-CSF, PMA, SCF, IgM, CD40L, anti-μ, H₂O₂, and T-cell modulators; anti-CD3, GM-CSF, PMA, 11-2 or anti-CD28.

### Nodes

In some embodiments, nodes are used in the classification, diagnosis, prognosis, theranosis, and/or prediction of an outcome of an autoimmune disease in a subject. As used herein, the term “node” describes a modulator and a molecule used to measure the activation level of an activatable element. For example, a node may be expressed in terms of [activatable element, modulator]. In some embodiments, a node can also incorporate marker and/or cell-type data, such as [activatable element, modulator, cell type]. In further embodiments, a node can describe the basal level of an activatable element measured in a cell type in the absence of a modulator, for example [response measured, basal, cell type]. In some of the embodiments discussed herein, a node comprises a modulator and a labeled antibody that binds to a state-specific epitope associated with an activatable element. “Node state data,” as used herein, refers to quantitative data corresponding to the signal of a molecule used to measure the response of an activatable element in one or more cells (i.e. a “node state”, “activation level”). Node state data may be raw signal data or metrics (“node state metrics”) quantifying any characteristic of the raw signal data. Node state metrics can express raw signal data as a relative value to a signal data generated from other cells (e.g. cells untreated with a modulator). A node can be any combination of an activatable element and a modulator. A node can also be any combination of an activatable element, modulator, and a cell type, wherein a cell type is determined by any of the preceding methods and may be expressed in terms of one or more markers.

### Binding Elements

Methods of the present invention may be used to detect any particular activatable element in a sample that is antigenically detectable and antigenically distinguishable from other activatable elements which are present in the sample. For example, the activation state-specific antibodies of the present invention can be used in the present methods to identify distinct signaling cascades of a subset or subpopulation of complex cell populations; and the ordering of protein activation (e g, kinase activation) in potential signaling hierarchies. Hence, in some embodiments the expression and phosphorylation of one or more polypeptides are detected and quantified using methods of the present invention. In some embodiments, the expression and phosphorylation of one or more polypeptides that are cellular components of a cellular pathway are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of an activatable element. Also preferably, the binding of the activation state-specific antibody is indicative of a specific activation state of a specific activatable element.

In some embodiments, the binding element is an antibody. In some embodiment, the binding element is an activation state-specific antibody.

The term “antibody” includes full length antibodies and antibody fragments, and may refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and posses other variations. See U.S. Ser. Nos. 61/048,886; 61/048,920 and 61/048,657 for more information about antibodies as binding elements.

Activation state specific antibodies can be used to detect kinase activity, however additional means for determining kinase activation are provided by the present invention. For example, substrates that are specifically recognized by protein kinases and phosphorylated thereby are known. Antibodies that specifically bind to such phosphorylated substrates but do not bind to such non-phosphorylated substrates (phospho-substrate antibodies) may be used to determine the presence of activated kinase in a sample.

The antigenicity of an activated isoform of an activatable element is distinguishable from the antigenicity of non-activated isoform of an activatable element or from the antigenicity of an isoform of a different activation state. In some embodiments, an activated isoform of an element possesses an epitope that is absent in a non-activated isoform of an element, or vice versa. In some embodiments, this difference is due to covalent addition of moieties to an element, such as phosphate moieties, or due to a structural change in an element, as through protein cleavage, or due to an otherwise induced conformational change in an element which causes the element to present the same sequence in an antigenically distinguishable way. In some embodiments, such a conformational change causes an activated isoform of an element to present at least one epitope that is not present in a non-activated isoform, or to not present at least one epitope that is presented by a non-activated isoform of the element. In some embodiments, the epitopes for the distinguishing antibodies are centered around the active site of the element, although as is known in the art, conformational changes in one area of an element may cause alterations in different areas of the element as well.

Many antibodies, many of which are commercially available (for example, see Cell Signaling Technology, www.cellsignal.com or Becton Dickinson, www.bd.com) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, FLT3 receptor, Kit receptor, FGF receptors, Eph receptors, Trk receptors, IGF receptors, Insulin receptor, Met receptor, Ret, VEGF receptors, TIE1, TIE2, erythropoietin receptor, thromobopoetin receptor, CD114, CD116, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, A™, ATR, phosphatases. Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPB, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pinl prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-FOXO, STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, elongation factors. In some embodiments, the protein is S6.

In some embodiments, an epitope-recognizing fragment of an activation state antibody rather than the whole antibody is used. In some embodiments, the epitope-recognizing fragment is immobilized. In some embodiments, the antibody light chain that recognizes an epitope is used. A recombinant nucleic acid encoding a light chain gene product that recognizes an epitope may be used to produce such an antibody fragment by recombinant means well known in the art.

In alternative embodiments of the instant invention, aromatic amino acids of protein binding elements may be replaced with other molecules. See U.S. Ser. Nos. 61/048,886; 61/048,920 and 61/048,657.

In some embodiments, the activation state-specific binding element is a peptide comprising a recognition structure that binds to a target structure on an activatable protein. A variety of recognition structures are well known in the art and can be made using methods known in the art, including by phage display libraries (see e.g., Gururaja et al. Chem. Biol. (2000) 7:515-27; Houimel et al., Eur. J. Immunol. (2001) 31:3535-45; Cochran et al. J. Am. Chem. Soc. (2001) 123:625-32; Houimel et al. Int. J. Cancer (2001) 92:748-55, each incorporated herein by reference). Further, fluorophores can be attached to such antibodies for use in the methods of the present invention.

A variety of recognition structures are known in the art (e.g., Cochran et al., J. Am. Chem. Soc. (2001) 123:625-32; Boer et al., Blood (2002) 100:467-73, each expressly incorporated herein by reference)) and can be produced using methods known in the art (see e.g., Boer et al., Blood (2002) 100:467-73; Gualillo et al., Mol. Cell. Endocrinol. (2002) 190:83-9, each expressly incorporated herein by reference)), including for example combinatorial chemistry methods for producing recognition structures such as polymers with affinity for a target structure on an activatable protein (see e.g., Barn et al., J. Comb. Chem. (2001) 3:534-41; Ju et al., Biotechnol. (1999) 64:232-9, each expressly incorporated herein by reference). In another embodiment, the activation state-specific antibody is a protein that only binds to an isoform of a specific activatable protein that is phosphorylated and does not bind to the isoform of this activatable protein when it is not phosphorylated or nonphosphorylated. In another embodiment the activation state-specific antibody is a protein that only binds to an isoform of an activatable protein that is intracellular and not extracellular, or vice versa. In a some embodiment, the recognition structure is an anti-laminin single-chain antibody fragment (scFv) (see e.g., Sanz et al., Gene Therapy (2002) 9:1049-53; Tse et al., J. Mol. Biol. (2002) 317:85-94, each expressly incorporated herein by reference).

In some embodiments the binding element is a nucleic acid. The term “nucleic acid” include nucleic acid analogs, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

In some embodiment the binding element is a small organic compound. Binding elements can be synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or binding elements that can then be used in the present invention.

In some embodiments the binding element is a carbohydrate. As used herein the term carbohydrate is meant to include any compound with the general formula (CH₂0)ₙ. Examples of carbohydrates are di-, tri- and oligosaccharides, as well polysaccharides such as glycogen, cellulose, and starches.

In some embodiments the binding element is a lipid. As used herein the term lipid herein is meant to include any water insoluble organic molecule that is soluble in nonpolar organic solvents. Examples of lipids are steroids, such as cholesterol, and phospholipids such as sphingomeylin.

Examples of activatable elements, activation states and methods of determining the activation level of activatable elements are described in US publication number 20060073474 entitled “Methods and compositions for detecting the activation state of multiple proteins in single cells” and US publication number 20050112700 entitled “Methods and compositions for risk stratification” the content of which are incorporate here by reference.

### Labels

The methods and compositions of the instant invention provide binding elements comprising a label or tag. By label is meant a molecule that can be directly (i.e., a primary label) or indirectly (i.e., a secondary label) detected; for example a label can be visualized and/or measured or otherwise identified so that its presence or absence can be known. Binding elements and labels for binding elements are shown in U.S. Ser. No. /048,886; 61/048,920 and 61/048,657.

A compound can be directly or indirectly conjugated to a label which provides a detectable signal, e.g. radioisotopes, fluorescers, enzymes, antibodies, particles such as magnetic particles, chemiluminescers, molecules that can be detected by mass spec, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes. In some embodiments of the invention, these labels may be conjugated to the binding elements.

In some embodiments, one or more binding elements are uniquely labeled. Using the example of two activation state specific antibodies, by “uniquely labeled” is meant that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels fall into four classes: a) isotopic labels, which may be radioactive or heavy isotopes; b) magnetic, electrical, thermal labels; c) colored, optical labels including luminescent, phosphorous and fluorescent dyes or moieties; and d) binding partners. Labels can also include enzymes (horseradish peroxidase, etc.) and magnetic particles. In some embodiments, the detection label is a primary label. A primary label is one that can be directly detected, such as a fluorophore.

Labels include optical labels such as fluorescent dyes or moieties. Fluorophores can be either “small molecule” fluors, or proteinaceous fluors (e.g. green fluorescent proteins and all variants thereof).

In some embodiments, activation state-specific antibodies are labeled with quantum dots as disclosed by Chattopadhyay, P. K. et al. Quantum dot semiconductor nanocrystals for immunophenotyping by polychromatic flow cytometry. Nat. Med. 12, 972-977 (2006). Quantum dot labels are commercially available through Invitrogen, http://probes.invitrogen.com/products/qdot/.

Quantum dot labeled antibodies can be used alone or they can be employed in conjunction with organic fluorochrome—conjugated antibodies to increase the total number of labels available. As the number of labeled antibodies increase so does the ability for subtyping known cell populations. Additionally, activation state-specific antibodies can be labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018,850, entitled Salicylamide-Lanthanide Complexes for Use as Luminescent Markers. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy.

In some embodiments, the activatable elements are labeled with tags suitable for Inductively Coupled Plasma Mass Spectrometer (ICP-MS) as disclosed in Tanner et al. Spectrochimica Acta Part B: Atomic Spectroscopy, 2007 March; 62(3): 188-195.

Alternatively, detection systems based on FRET, discussed in detail below, may be used. FRET finds use in the instant invention, for example, in detecting activation states that involve clustering or multimerization wherein the proximity of two FRET labels is altered due to activation. In some embodiments, at least two fluorescent labels are used which are members of a fluorescence resonance energy transfer (FRET) pair.

The methods and composition of the present invention may also make use of label enzymes. By label enzyme is meant an enzyme that may be reacted in the presence of a label enzyme substrate that produces a detectable product. Suitable label enzymes for use in the present invention include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By radioisotope is meant any radioactive molecule. Suitable radioisotopes for use in the invention include, but are not limited to ¹⁴C, ³H, ³²P, ³³P, ³⁵S, ¹²⁵I and ¹³¹I. The use of radioisotopes as labels is well known in the art.

As mentioned, labels may be indirectly detected, that is, the tag is a partner of a binding pair. By “partner of a binding pair” is meant one of a first and a second moiety, wherein the first and the second moiety have a specific binding affinity for each other. Suitable binding pairs for use in the invention include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255: 192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto. As will be appreciated by those in the art, binding pair partners may be used in applications other than for labeling, as is described herein.

As will be appreciated by those in the art, a partner of one binding pair may also be a partner of another binding pair. For example, an antigen (first moiety) may bind to a first antibody (second moiety) that may, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair may comprise a label, as described above. It will further be appreciated that this allows for a tag to be indirectly labeled upon the binding of a binding partner comprising a label. Attaching a label to a tag that is a partner of a binding pair, as just described, is referred to herein as “indirect labeling”.

By “surface substrate binding molecule” or “attachment tag” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate [Field et al., Mol. Cell. Biol., 8:2159-2165 (1988)]; the c-myc tag and the 8F9, 3C7, 6E10, G4, B7 and 9E10 antibody substrates thereto [Evan et al., Molecular and Cellular Biology, 5:3610-3616 (1985)]; and the Herpes Simplex virus glycoprotein D (gD) tag and its antibody substrate [Paborsky et al., Protein Engineering, 3(6):547-553 (1990)]. In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

An alternative activation state indicator useful with the instant invention is one that allows for the detection of activation by indicating the result of such activation. For example, phosphorylation of a substrate can be used to detect the activation of the kinase responsible for phosphorylating that substrate. Similarly, cleavage of a substrate can be used as an indicator of the activation of a protease responsible for such cleavage. Methods are well known in the art that allow coupling of such indications to detectable signals, such as the labels and tags described above in connection with binding elements. For example, cleavage of a substrate can result in the removal of a quenching moiety and thus allowing for a detectable signal being produced from a previously quenched label.

### Drug Transporters

A key issue in the treatment of many cancers is the development of resistance to chemotherapeutic drugs. Of the many resistance mechanisms, two classes of transporters play a major role. Of the many resistance mechanisms, two classes of transporters play a major role: 1) human ATP-binding cassette (ABC) superfamily of proteins; 2) Concentrative and Equilibrative Nucleoside Transporters (CNT and ENT, respectively). For further discussion, see U.S. Patent Application 61/085,789.

### Gating

In some embodiments of the invention, different gating strategies can be used in order to analyze only blasts in the sample of mixed population after treatment with the modulator. These gating strategies can be based on the presence of one or more specific surface marker expressed on each cell type. See U.S. Patent Applications 61/085,789, 61/120,320, and 61/079,766, hereby incorporated by reference.

### Detection

In practicing the methods of this invention, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention is performed according to standard techniques and protocols well-established in the art.

One or more activatable elements can be detected and/or quantified by any method that detect and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See the above patents and applications for example methods.

In some embodiments, the present invention provides methods for determining an activatable element's activation profile for a single cell. The methods may comprise analyzing cells by flow cytometry on the basis of the activation level of at least two activatable elements. Binding elements (e.g. activation state-specific antibodies) are used to analyze cells on the basis of activatable element activation level, and can be detected as described below. Alternatively, non-binding elements systems as described above can be used in any system described herein.

Detection of cell signaling states may be accomplished using binding elements and labels. Cell signaling states may be detected by a variety of methods known in the art. They generally involve a binding element, such as an antibody, and a label, such as a fluorochrome to form a detection element. Detection elements do not need to have both of the above agents, but can be one unit that possesses both qualities. These and other methods are well described in U.S. Pat. Nos. 7,381,535 and 7,393,656 and U.S. Ser. Nos. 10/193,462; 11/655,785; 11/655,789; 11/655,821; 11/338,957, 61/048,886; 61/048,920; and 61/048,657 which are all incorporated by reference in their entireties.

In one embodiment of the invention, it is advantageous to increase the signal to noise ratio by contacting the cells with the antibody and label for a time greater than 5, 6, 7, 8, 9, 10, 11, 12, 13, 14, 15, 16, 17, 18, 19, 20, 24 or up to 48 or more hours.

When using fluorescent labeled components in the methods and compositions of the present invention, it will recognized that different types of fluorescent monitoring systems, e.g., cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation level in an activatable element in response to a modulator. Other flow cytometers that are commercially available include the LSR II and the Canto II both available from Becton Dickinson. See Shapiro, Howard M., Practical Flow Cytometry, 4th Ed., John Wiley & Sons, Inc., 2003 for additional information on flow cytometers.

In some embodiments, the cells are first contacted with fluorescent-labeled activation state-specific binding elements (e.g. antibodies) directed against specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety. See the patents, applications and articles referred to, and incorporated above for detection systems.

Fluorescent compounds such as Daunorubicin and Enzastaurin are problematic for flow cytometry based biological assays due to their broad fluorescence emission spectra. These compounds get trapped inside cells after fixation with agents like paraformaldehyde, and are excited by one or more of the lasers found on flow cytometers. The fluorescence emission of these compounds is often detected in multiple PMT detectors which complicates their use in multiparametric flow cytometry. A way to get around this problem is to compensate out the fluorescence emission of the compound from the PMT detectors used to measure the relevant biological markers. This is achieved using a PMT detector with a bandpass filter near the emission maximum of the fluorescent compound, and cells incubated with the compound as the compensation control when calculating a compensation matrix. The cells incubated with the fluorescent compound are fixed with paraformaldehyde, then washed and permeabilized with 100% methanol. The methanol is washed out and the cells are mixed with unlabeled fixed/permed cells to yield a compensation control consisting of a mixture of fluorescent and negative cell populations.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific binding element. The cell-binding element-particle complex can then be physically separated from non-positive or non-labeled cells, for example, using a magnetic field. When using magnetically responsive particles, the positive or labeled cells can be retained in a container using a magnetic field while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyze the population of cells by flow cytometry. Preferably, cells are analyzed on the basis of the activation level of at least two activatable elements. In some embodiments, a multiplicity of activatable element activation-state antibodies is used to simultaneously determine the activation level of a multiplicity of elements.

In some embodiment, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

As will be appreciated, the present invention also provides for the ordering of element clustering events in signal transduction. Particularly, the present invention allows the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

The present invention provides a valuable method of determining the presence of cellular subsets within cellular populations. Ideally, signal transduction pathways are evaluated in homogeneous cell populations to ensure that variances in signaling between cells do not qualitatively nor quantitatively mask signal transduction events and alterations therein. As the ultimate homogeneous system is the single cell, the present invention allows the individual evaluation of cells to allow true differences to be identified in a significant way.

Thus, the invention provides methods of distinguishing cellular subsets within a larger cellular population. As outlined herein, these cellular subsets often exhibit altered biological characteristics (e.g. activation levels, altered response to modulators) as compared to other subsets within the population. For example, as outlined herein, the methods of the invention allow the identification of subsets of cells from a population such as primary cell populations, e.g. peripheral blood mononuclear cells that exhibit altered responses (e.g. response associated with presence of a condition) as compared to other subsets. In addition, this type of evaluation distinguishes between different activation states, altered responses to modulators, cell lineages, cell differentiation states, etc.

As will be appreciated, these methods provide for the identification of distinct signaling cascades for both artificial and stimulatory conditions in complex cell populations, such as peripheral blood mononuclear cells, or naive and memory lymphocytes.

When necessary cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 min in acetone at −200 C; and the like as known in the art and according to the methods described herein.

In some embodiments, one or more cells are contained in a well of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activativatable. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, 2007 March; 62(3): 188-195.).

As will be appreciated by one of skill in the art, the instant methods and compositions find use in a variety of other assay formats in addition to flow cytometry analysis. For example, DNA microarrays are commercially available through a variety of sources (Affymetrix, Santa Clara, Calif.) or they can be custom made in the lab using arrayers which are also know (Perkin Elmer). In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell.

In some embodiments, a chip comprises a multiplicity of the “second set binding elements,” in this case generally unlabeled. Such a chip is contacted with sample, preferably cell extract, and a second multiplicity of binding elements comprising element activation state specific binding elements is used in the sandwich assay to simultaneously determine the presence of a multiplicity of activated elements in sample. Preferably, each of the multiplicity of activation state-specific binding elements is uniquely labeled to facilitate detection.

In some embodiments, confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell® plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described below.

In some embodiments, the detecting is by high pressure liquid chromatography (HPLC), for example, reverse phase HPLC, and in a further aspect, the detecting is by mass spectrometry.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiment, the methods of the invention include the use of liquid handling components. The liquid handling systems can include robotic systems comprising any number of components. In addition, any or all of the steps outlined herein may be automated; thus, for example, the systems may be completely or partially automated. See U.S. Patent Application No. 61/048,657.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc.

### Analysis

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g. microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. See Krutzik et al, Nature Chemical Biology February 2008. Methods for the analysis of multiple parameters are well known in the art. See U.S. Patent Application No. 61/079,579 for gating analysis.

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. Flow cytometry experiments ideally include a reference sample against which experimental samples are compared. Reference samples can include normal and/or cells associated with a condition (e.g. tumor cells). See also U.S. Patent Application No. 61/079,537 for visualization tools.

### Kits

In some embodiments the invention provides kits. Kits provided by the invention may comprise one or more of the state-specific binding elements described herein, such as phosphospecific antibodies. A kit may also include other reagents that are useful in the invention, such as modulators, fixatives, containers, plates, buffers, therapeutic agents, instructions, and the like.

In some embodiments, the kit can comprise one or more modulators, such as those listed above. In some instances, the kit can be used to distinguish modulators that are specific for the PI3K pathway, mTOR pathway, or modulate both the PI3K and mTOR pathway. In some embodiments, the kit can also be used to distinguish modulators that are specific for the above pathways and characterize side effects associated with the modulator, or each of the above stated pathways.

In some embodiments, the kit comprises one or more of the antibodies that recognize epitopes within but not limited to specific domains of the following PI3-Kinase components, fragments and variations thereof: p85 adaptor family (including p85α, p85β, p55α, p55β, p50α), p110α, p110β, p110δ; antibodies for the phosphorylated or non phosphohorylated molecules listed below: epitopes within PDK-1; Akt isoforms; PRAS40; Mdm2; TSC2; GSK3β; BAD; FOXO transcription factors; NFkappaB; mTor; p70S6 kinase; ribosomal S6; and 4EBP-1.

In some embodiments, the kit comprises one or more antibodies that recognize non-phospho and phospho epitopes within a protein, including, but not limited to Lnk, SOCS3, SH2-B, Mpl, Epo receptor, and Flt-3 receptor. Kits may also include instructions for use and software to plan, track experiments, and files which contain information to help run experiments.

Kits provided by the invention may comprise one or more of the modulators described herein.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

Examples that may serve to more fully describe the manner of using the above-described invention, as well as to set forth the best modes contemplated for carrying out various aspects of the invention can be seen in the incorporated application 61/120,320. It is understood that these examples in no way serve to limit the true scope of this invention, but rather are presented for illustrative purposes. All references cited herein are expressly incorporated by reference in their entireties.

An experiment can be designed to develop a pharmacodynamic (PD) assay as a marker for tumor cells that will monitor GDC-0941 in clinical trials for multiple myeloma and AML, and to evaluate whether p-Akt levels alone or in combination with other PI3K and/or mTOR pathway markers represent a reliable PD assay to monitor P110 inhibition. The experiment would compare metrics for data analysis, such as: fold change of p-Akt comparing levels untreated versus drug-treated cells, fold change of total phospho-Akt in untreated versus drug-treated cells, and fold change of p-Akt expressed as a ratio of p-Akt to total Akt in untreated versus drug-treated cells.

The experiment would use cell lines to identify optimal pathway profile (combination of nodes in PI3 Kinase pathway) and experimental conditions to measure P110 inhibition in cancer cell lines. It would use leukemic cell lines U937, THP and KG-1 and myeloma cell lines OPM2, MM1, and ESM. The phosphosignaling nodes to be analyzed include: p-Akt (pS⁴⁷³), p-BAD (pS136), PRAS-40 (pT²⁴⁶), and p-S6 (pS^{235/236}). The modulators to be used include: for leukemic cell lines—no modulator (control), SCF, F1t3L, G-CSF, and IGF-1; for myeloma cell lines—no modulator (control), SCF, F1t3L, IL-6, and IGF-1; and a range of serum starvation conditions will also be tested in order to determine the optimal dynamic range for evaluating P110 inhibition. Compounds to be tested include GDC0-0941—to be titrated and based upon conditions optimized from preclinical work.

In addition to cell line data, whole blood can be used to test the application of the phosphoprotein profile as a pharmacodynamic marker in whole blood, the likely surrogate tissue to be used in clinical studies. The most robust activation of the PI3 kinase pathway can be seen in B lymphocytes and also in T cells in response to the modulators described below.

The tissue type is whole blood or cryopreserved peripheral blood mononuclear cells (PBMCs). The phosphoproteins to be analyzed are the same as in Cell Line Study (i.e. p-Akt, p-BAD, PRAS-40, and pS6). The modulators include: B-cell modulators, CD40L, anti-μ, H₂0₂, and no modulator (control) and T-cell modulators; anti-CD3, anti-CD28, and no modulator. The cell types to be studied are B-cells and the compound to be tested is GDC-0941.

A further experiment can be designed to correlate the whole blood PD assay above to a xenograft murine model. Another experiment can be designed to use bone marrow mononuclear cells (BMMC) taken from AML Patients treated with GDC-0941 and the assay conditions noted above.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A method for classifying a cell comprising:
contacting the cell with a PI3K and/or mTOR pathway modulator;
determining the presence or absence of a change in activation level of an activatable element in the cell; and
classifying the cell based on the presence or absence of the change in the activation level of the activatable element.

2. The method of claim 1, wherein the change in activation level of the activatable element is an increase in activation level of the activatable element.

3. The method of claim 1, wherein the cell is a cancer cell or hematopoietic cell.

4. The method of claim 1, wherein the presence or absence of a change in the activation level of the activatable element is compared to a normal cell contacted with the PI3K and/or mTOR inhibitor.

5. The method of claim 1, wherein the presence or absence of a change in the activation levels of the activatable element is determined in the determining step.

6. The method of claim 1, wherein the classification comprises classifying the cell as a cell that is correlated with a clinical outcome.

7. The method of claim 6, wherein the clinical outcome is the presence or absence of a cancer, metabolic disorder or immune disorder.

8. The method of claim 6, wherein the clinical outcome is the staging or grading of a neoplastic condition.

9. The method of claim 1, wherein the classification further comprises determining a method of treatment.

10. The method of claim 1, wherein the modulator is a cancer cell modulator or hematopoietic cell modulator.

11. The method of claim 1, wherein the modulator is a growth factor, chemokine, cytokine, drug, immune modulator, ion, neurotransmitter, adhesion molecule, hormone, small molecule, inorganic compound, polynucleotide, antibody, natural compound, lectin, lactone, chemotherapeutic agent, biological response modifier, carbohydrate, protease, free radical, complex and undefined biologic composition, cellular secretion, glandular secretion, physiologic fluid, reactive oxygen species, virus, electromagnetic radiation, ultraviolet radiation, infrared radiation, particulate radiation, redox potential, pH modifier, the presence or absences of a nutrient, change in temperature, change in oxygen partial pressure, change in ion concentration or application of oxidative stress.

12. The method of claim 1, wherein the PI3K and/or mTOR pathway modulator is a PI3K and/or mTOR pathway inhibitor is a therapeutic agent.

13. The method of claim 12, wherein the inhibitor is a therapeutic agent.

14. The method of claim 1, wherein the activatable element is a PI3K pathway protein.

15. The method of claim 14, wherein the PI3K pathway protein is PI3K, p110 isoforms, PDK-1, Akt isoforms, PRAS40, Mdm2, TSC2, GSK3β, BAD, FOXO transcription factors, NFkB, mTOR, p70S6 kinase, Ribosomal S6, 4EBP1, Paxillin, PKCα, PKCβ, SGK, TSC1, Rictor or Raptor.

16. The method of claim 1, further comprising analyzing expression level of the PI3K pathway protein.

17. The method of claim 1, wherein the cell is from a patient sample.

18. The method of claim 17, further comprising determining a clinical outcome based on the correlation of the activity of the PI3K regulatory protein with the activity of the PI3K pathway component.

19. The method of claim 18, further comprising determining a method of treatment of the patient based on the correlation of the activity of the PI3K regulatory protein with the activity of the PI3K pathway component.

20. A method of determining the presence or absence of a condition in an individual comprising:
subjecting a cell from the individual to a PI3k and/or mTOR pathway inhibitor;
determining the activation level of an activatable element in the cell; and
determining the presence or absence of the condition based on the activation level.

21. A method of correlating and/or classifying an activatable state of a cancer cell with a clinical outcome in an individual comprising:
subjecting the cancer cell from the individual to a PI3k and/or mTOR pathway modulator;
determining the activation level of an activatable element; and
identifying a pattern of the activation level of the activatable element to determine the presence or absence of an alteration in signaling, wherein the presence of the alteration is indicative of a clinical outcome.
